SEQUENCE LISTING (114 sequences)

SEQ ID NO: 1 - DNA, 7785 bp, Hepatitis C virus
  MISC_FEATURE (1)..(341): 5′UTR
gccagccccctgatgggggcgacactccaccatagatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaacctaacaccaaccgccgcccacaggacgtcaagttcccgggcggtggtcagatcgttggtggagtttacctgttgccgcgcaggggcccccggttgggtgtgcgcgcgactaggaagacttccgagcggtcgcaacctcgtggaaggcgacaacctatccccaaggctcgccggcccgagggcagggcctgggctcagcccgggtacccctggcccctctatggcaatgagggcttagggtgggcaggatggctcctgtcaccccgcggctctcggcctagttggggccccacggacccccggcgtaggtcgcgtaacttgggtaaggtcatcgataccctcacatgcggcttcgccgacctcatggggtacattccgctcgtcggtgcccccctagggggcgctgccagggccctagcacatggtgtccgggttctggaggacggcgtgaactacgcaacagggaatttgcccggttgctctttctctatcttcctcttggctctgctgtcctgtctgaccatcccagcttccgcttatgaagtgcgcaacgtgtccggaatataccatgtcacgaacgactgctccaactcaagcattgtgtatgaggcagcggacgtgatcatgcatacccccgggtgcgtgccctgtgttcgggagggtaacgcctcccgctgttgggcagcgctcactcccacgctcgcggtcgggaatgccagcgtccccactaaggcaatacggcgccacgtcgatctgcttgttgggacggctgctttctgctccgccatgtacgtgggggatctctgcggatacatcaccaaactcctgctcgccacactcggtctgctcatggtgctccaggctgccatagctagggtgccgtacttcgtacgcactcaggggctcattcgtgtgtgtatgttagtgcggaaagtcgccgggggtcactatgcccagatggccttcatcaagctggccgcactgacaggtacatacgtttatgaccatcttactccactgcgagattgggcccatgcgggcctgcgagaccttgcggtggcagtggagcccgtcatcttctctgacatggagaccaagatcatcacctggggagcagacaccgcggcgtgtggggatattattttgggtctgcccgtctccgcccgaagggggagggagatacttctggggccggccgatagtcttgaggggcgggggtggcgactccttgcgcccatcacggcttattctcaacagacgcggggtttactcggctgcatcatcactagtctcacgggccgggacaagaaccaggtcgagggggaggttcaagtggtttcgaccgcgacacaatccttcctggcgacctgtgtcaacggcgtgtgttggactgtctatcatggtgccggctcaaaaaccctagccggcccaaaagggccgattatccaaatgtataccaatgtagaccaggaccttgttggctggcaagcgccccccggggcgcgttccttgacaccatgcacctgcggcagctcggacctttacctggttacgagacatgctgacgtcattccggtgcgccggcgaggtgacggtagggggagcctactctcccccaaacccatctcctacttgaaaggctcttcgggtggtccgctgctctgcccttcggggcacgctgtgggcatctttcgggctgctgtgtgcacccgggggattgcgaaggctgtggactttgtacccgttgagtgtatggaaactactatgcggtctccggtcttcacagacaactcgtcccccccgaccgtaccgcagacattccaagtggcccatctacacgctcccactggcagcggcaaaagcaccaaagtaccggctgcatatgcggcccaagggtataaggtactcgtcctgaacccgtccgttgccgccaccctgagttttggggcgtatatgtccaaggcacatggtgtcgaccctaacatcagaactgggatgaggaccatcaccacaggcgctcccatcacgtactccacctatggcaagttccttgccgacggtggttgttccgggggcgcctatgacatcatattatgtgatgagtgccactcaactgactcaactactgttttaggcatcggcacagttctggaccaagcggagacggctggagcgcgactcgtcgtgctcgccaccgctacgcctccaggatcggtcaccgtgccacaccccaatatcgaggaagtggctctgtccaacactggagagatccccttctatggcaaagccatccctatcgaggtcatcaaggggggaaggcatctcattttctgtcattccaagaagaaatgcgacgagcttgctgcaaagttgtcaggtctcggactcaatgctgtagtgtattaccggggccttgacgtgtccgtcatacctaccagcggagacgtcgttgtcgtggcaacagacgctctaatgacgggctataccggtgactttgactcagtgatcgactgtaatacatgtgtcactcagacagtcgacttcagcttggatcctaccttcaccattgacacgacgaccgtaccccaagacgcggtatcacgctcgcagcggcgaggtaggactggcaggggtaggggaggcatctacaggtttgtgactccaggagaacggccctcgggcatgttcgattcttcggtcttgtgtgagtgttatgacgcgggctgtgcttggtatgagctcacgcccgccgaaaccacggttaggttgcgggcttaccttaatacaccagggttgcccgtctgtcaggaccacctggagttctgggagggtgtcttcacaggcctcacccacatagaagctcatctcttgtcccagactaaggatgcaggagacaattacccctacctggtagcgtaccaagccacggtgtgcgccagggctcaggccccacctccgtcttgggatcaaatgtggaagtgtctcatgcggcttaaacctacgctgcacgggccaacacccctgctgtataggctaggagccgtccagaatgaggtcacccttacacaccccataaccaaatacatcatcacatgcatgtcagctgacctggaggttgtcactagcacctgggtgctagtaggcggagtccttgcagctttggccgcatactgcctgacaacaggcagtgtggtcattgtgggcaggatcatcttgtccgggaagccggctgtcatccccgacagggaagtcctctaccaggcgttcgatgaaatggaggagtgtgcctcacacctcccttacatcgaacagggaatgcagctcgccgagcaattcaagcagaaagcgctcgggctgctacaaacggccactaagcaagcggaggctgctgctcccatggtggagtccaaatggcacgcccttgaggctttctgggcgaagcacatgtggaacttcatcagcgggatacagtacttagcaggcttgtccactctgcctgggaaccccgcaatagcatcactgatggcattcacagcctctgtcaccagcccgcttaccacccagagcaccctcttgtttaacatcttggggggatgggtggctgcccaactcgctccccccggtgctgcttcggcttttgtgggcgccggaattgccggcgcggccgtaggcagcataggccttgggaaggtgcttgtggacattctggctggatatggggcaggggtggcaggcgcactcgtggcttttaagatcatgagcggcgatatgccctccaccgaggacctggttaacttgcttcctgccatcctctctcctggtgccctggtcgtcggggtcgtgtgcgcagcaatactgcgtcggcacgtgggcccgggagagggggctgtgcagtggatgaaccggctgatagcgttcgcttcccggggtaaccacatctcccccacgcactatgtgcctgagagcgacgccgcagcgcgtgttacccagattctttccaaccttaccatcactcagctgctgaagaggcttcaccaatggatcaatgaggactgctccacgccatgctccggctcgtggcttagggatgtttgggactggatatgcacggtgttggctgacttcaagacctggctccagtccaagctcctgccgcggttgccgggagtccctttcttctcatgccaacgcgggtacaagggagtttggcggggggatggcatgatgcataccacctgcccatgtggagcacaaatcaccggacatgtcaaaaatggttccatgaggatcgctgggcctagaacctgcagcaacacgtggcatgggacgttccccatcaacgcatacaccacgggcccctgcacaccctccccggcgcccaactattccaaggcgctatggcgggtggctgctgaggagtacgtggaagttacgcgagtgggagacttccactacgtgacgggcatgaccactgacaacataaaatgcccatgccaggttccggcccccgaattcttcacagaactggatggagtgcggttgcacaggtacgctccggtgtgcaaacccctcctacgggaggaggttttattccaggttgggtgcaaccaatacctggtcgggtcacagcttccatgcgagcccgaaccggacgtagcagtgctcacttccatgcttgccgacccctcccacattacagcagagacagctaagcgtaggttggccagggggtctcccccctccttggccagctcgtcagctagccagttgtctgcaccttctttgaaggcgacatgcaatacccatcaccgctccccggaccttgacctcatcgaggccaacctcctgtggtggcaggagaagggtggaaacatcacccgtgtggagtcagagaacaaggtgataatcatggactctttcgatccgcttcgagcggaggaggatgagagggaaatatctgttgcggcggagatcctgcggcaatccaggaaattccccccagcgttgcccgtatgggcacgcccggattataaccctccactactagagccctggaaggacccggactatgtccctccggtggtacatgggtgcccgctgccgcctgccaagactcctccaataccacctccacggaggaaaaggacggttgtcctgacagagtccaccgtgtcttctgttctggcggagctcactactaagaccttcggcagctccgaatcgtcggccgctgatagcggcatggcgaccgcccctcctgaccaggcctccggcgacggcgacaaagagtccgacgttgagtcgtactcctccatgcccccccttgagggagagccgggggaccccgatctcagcgacgggtcttggtctaccgtgagcgaggaggctggtgaggacgtcgtctgctgttcaatgtcctatacatggacaggcgccttgatcacaccatgcgctgcggaggaaagcaagctgcccatcaacgcgttgagcaactctttgctgcgtcatcacaacatggtctatgccacaacatctcgcagcgcaagccagcggcagaagaaggtcacctttgacagactgcaggtcctggatgatcactaccgggacgtgcttaaggagatgaaggcgaaggcgtccacagttaaggctaaacttctctctgtagaagaagcctgcaagctgacgcccccacattcggccaaatctaagtttggttatggggcaaaggacgtccggaacctatccagcagggccgttaaccacattcgctccgtgtggaaggacttgctggaagacactgaaacaccaattgacaccaccatcatggcaaaaagtgaggttttctgcatccaaccagagaaaggaggccgcaagccagctcgccttatcgtgttcccagacctgggagtccgtgtatgcgagaaaatggccctctacgacgtggtctccacccttcctcaggccgtgatgggctcctcatatggattccaatactctcctgggcagcgagtcgagttcctggtaaatgcctggaaatcaaagaaaaaccccatgggcttctcatatgacactcgctgtttcgactcaacggtcactgagagtgacatccgcgttgaggagtcaatctaccaatgttgtgacttggcccccgaagccagacaggccataaagtcgctcacagagcggctctatatcgggggtcccctgactaattcaaaagggcaaaactgcggttatcgccggtgtcgcgccagcggcgtgctgacgactagctgcggtaataccctcacatgttacttgaaggccgctgcggcctgtcgagctgcgaagctccaggactgcacgatgctcgtgaacggagacgacctagtcgttatctgtgagagtgcgggaacccaagaggatgcggcgaacctacgagtcttcacggaggctatgactaggtactctgctcccccaggggactcgcctcaaccagaatacgacttggagttgataacatcttgctcctccaatgtgtcggtcgcgcacgatgcgtctggcaagagggtgtactacctcactcgtgaccccaccaccccccttgcacgggctgcgtgggagacagctagacacactccagtcaactcctggctaggcaatatcatcatgtatgcgcccaccttatgggcaaggatgattctgatgacccacttcttctccatccttctagctcaggaacaacttggaaaagccctggattgccagatctatggggcctgttactccattgagccacttgatctacctcagatcattgaacgactccacggtcttagcgcattttcactccatagttactctccaggtgagatcaatagggtggcttcatgcctcaggaaacttggggtaccacccttgcgagtctggagacatcgggccagaagtgtccgcgctaagctactgtcccagggggggagggccgccacttgtggcaagtacctcttcaactgggcagtaaagaccaagcttaaactcactccaatcccggctgcgtcccagttggatttatccagctggttcgttgctggttacagcgggggagacatatatcacagcctgtctcgtgcccgaccccgctggttcatgtggtgcctactcctactttctgtaggggtaggcatctatctactccccaaccgatgaacggggagctaaacactccaggccaataggccatcctgttttttttttcttttttttttttcctttttttttttttttttttttttttccttttttttttttttcttttttccttttctttcctttggtggctccatcttagccctagtcacggctagctgtgaaaggtccgtgagccgcttgactgcagagagtgctgatactggcctctctgcagatcatg

SEQ ID NO: 2 - protein, 203 aa, Hepatitis C virus
MSTNPKPQRKTKPNTNRRPQDVKFPGGGQIVGGVYLLPRRGPRLGVRATRKTSERSQPRGRRQPIPKARRPEGRAWAQPGYPWPLYGNEGLGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTIPASAYEVRNVSGIYHV

SEQ ID NO: 3 - protein, 219 aa, Hepatitis C virus
TNDCSNSSIVYEAADVIMHTPGCVPCVREGNASRCWAALTPTLAVGNASVPTKAIRRHVDLLVGTAAFCSAMYVGDLCGYITKLLLATLGLLMVLQAAIARVPYFVRTQGLIRVCMLVRKVAGGHYAQMAFIKLAALTGTYVYDHLTPLRDWAHAGLRDLAVAVEPVIFSDMETKIITWGADTAACGDIILGLPVSARRGREILLGPADSLEGRGWRLL

SEQ ID NO: 4 - protein, 631 aa, Hepatitis C virus
APITAYSQQTRGLLGCIITSLTGRDKNQVEGEVQVVSTATQSFLATCVNGVCWTVYHGAGSKTLAGPKGPIIQMYTNVDQDLVGWQAPPGARSLTPCTCGSSDLYLVTRHADVIPVRRRGDGRGSLLSPKPISYLKGSSGGPLLCPSGHAVGIFRAAVCTRGIAKAVDFVPVECMETTMRSPVFTDNSSPPTVPQTFQVAHLHAPTGSGKSTKVPAAYAAQGYKVLVLNPSVAATLSFGAYMSKAHGVDPNIRTGMRTITTGAPITYSTYGKFLADGGCSGGAYDIILCDECHSTDSTTVLGIGTVLDQAETAGARLVVLATATPPGSVTVPHPNIEEVALSNTGEIPFYGKAIPIEVIKGGRHLIFCHSKKKCDELAAKLSGLGLNAVVYYRGLDVSVIPTSGDVVVVATDALMTGYTGDFDSVIDCNTCVTQTVDFSLDPTFTIDTTTVPQDAVSRSQRRGRTGRGRGGIYRFVTPGERPSGMFDSSVLCECYDAGCAWYELTPAETTVRLRAYLNTPGLPVCQDHLEFWEGVFTGLTHIEAHLLSQTKDAGDNYPYLVAYQATVCARAQAPPPSWDQMWKCLMRLKPTLHGPTPLLYRLGAVQNEVTLTHPITKYIITCMSADLEVVT

SEQ ID NO: 5 - protein, 54 aa, Hepatitis C virus
STWVLVGGVLAALAAYCLTTGSVVIVGRIILSGKPAVIPDREVLYQAFDEMEEC

SEQ ID NO: 6 - protein, 260 aa, Hepatitis C virus
ASHLPYIEQGMQLAEQFKQKALGLLQTATKQAEAAAPMVESKWHALEAFWAKHMWNFISGIQYLAGLSTLPGNPAIASLMAFTASVTSPLTTQSTLLFNILGGWVAAQLAPPGAASAFVGAGIAGAAVGSIGLGKVLVDILAGYGAGVAGALVAFKIMSGDMPSTEDLVNLLPAILSPGALVVGVVCAAILRRHVGPGEGAVQWMNRLIAFASRGNHISPTHYVPESDAAARVTQILSNLTITQLLKRLHQWINEDCSTP

SEQ ID NO: 7 - protein, 448 aa, Hepatitis C virus
CSGSWLRDVWDWICTVLADFKTWLQSKLLPRLPGVPFFSCQRGYKGVWRGDGMMHTTCPCGAQITGHVKNGSMRIAGPRTCSNTWHGTFPINAYTTGPCTPSPAPNYSKALWRVAAEEYVEVTRVGDFHYVTGMTTDNIKCPCQVPAPEFFTELDGVRLHRYAPVCKPLLREEVLFQVGCNQYLVGSQLPCEPEPDVAVLTSMLADPSHITAETAKRRLARGSPPSLASSSASQLSAPSLKATCNTHHRSPDLDLIEANLLWWQEKGGNITRVESENKVIIMDSFDPLRAEEDEREISVAAEILRQSRKFPPALPVWARPDYNPPLLEPWKDPDYVPPVVHGCPLPPAKTPPIPPPRRKRTVVLTESTVSSVLAELTTKTFGSSESSAADSGMATAPPDQASGDGDKESDVESYSSMPPLEGEPGDPDLSDGSWSTVSEEAGEDVVCC

SEQ ID NO: 8 - protein, 591 aa, Hepatitis C virus
SMSYTWTGALITPCAAEESKLPINALSNSLLRHHNMVYATTSRSASQRQKKVTFDRLQVLDDHYRDVLKEMKAKASTVKAKLLSVEEACKLTPPHSAKSKFGYGAKDVRNLSSRAVNHIRSVWKDLLEDTETPIDTTIMAKSEVFCIQPEKGGRKPARLIVFPDLGVRVCEKMALYDVVSTLPQAVMGSSYGFQYSPGQRVEFLVNAWKSKKNPMGFSYDTRCFDSTVTESDIRVEESIYQCCDLAPEARQAIKSLTERLYIGGPLTNSKGQNCGYRRCRASGVLTTSCGNTLTCYLKAAAACRAAKLQDCTMLVNGDDLVVICESAGTQEDAANLRVFTEAMTRYSAPPGDSPQPEYDLELITSCSSNVSVAHDASGKRVYYLTRDPTTPLARAAWETARHTPVNSWLGNIIMYAPTLWARMILMTHFFSILLAQEQLGKALDCQIYGACYSIEPLDLPQIIERLHGLSAFSLHSYSPGEINRVASCLRKLGVPPLRVWRHRARSVRAKLLSQGGRAATCGKYLFNWAVKTKLKLTPIPAASQLDLSSWFVAGYSGGDIYHSLSRARPRWFMWCLLLLSVGVGIYLLPNR

SEQ ID NO: 9 - DNA, 1060 bp, Hepatitis C virus
  CDS (229)..(1059)
ccaggcccccccctcccgggagagccatagtggtctgcggaaccggtgagtacaccggaattaccggaaagactgggtcctttcttggataaacccactctatgtccggtcatttgggcgtgcccccgcaagactgctagccgagtagcgttggggtgcgaagggccttgtggtactgcctgatagggtgcttgcgagtgccccgggaggtctcgtagaccgtgcatcatgagcacaaatcctaaacctcaaagaaaaaccaaaagaagcacaaaccgccgcccacaggacgtcaagttcccgggtggcggtcagatcgttggcggagtttacttgctgccgcgcaggggccccaggttgggtgtgcgcgcgacaaggaagacttccgagcgatcccagccgcgtgggagacgccagcccatcccgaaagatcggcgctccaccggcaagtcctggggaaagccaggatatccttggcccctgtatggaaacgagggttgcggctgggcaggttggctcctgtccccccgcgggtctcgtcctacttggggccccaccgacccccggcacagatcacgcaattggggtaaagtcatcgatacccttacgtgtggttttgccgacctcatggggtacatccctgtcattggcgccccggtcggaggcgttgccagagccctagcgcacggtgttagggtcctggaagacggggtgaattacgcaacagggaatctacccggttgctctttttctatcttcttgcttgcccttctgtcgtgcgtcacagtgccagtgtctgcagtggaggtcaggaacattagttctagctactatgccactaacgattgctcggacaacagcatcacctggcagcgccttgtgtttgaagtcacaaaatggttgttagcaatcctggggtctgcccacctccttaaagcgtccctgctacgggtgccatactttgtgagggctcacgctctgctacgggtgtgtaccctggtgaggcaccttgcaggagctaagtacatccagatgctgttgatcactgtgggcaggcgga

SEQ ID NO: 10 - protein, 277 aa, Hepatitis C virus
MSTNPKPQRKTKRSTNRRPQDVKFPGGGQIVGGVYLLPRRGPRLGVRATRKTSERSQPRGRRQPIPKDRRSTGKSWGKPGYPWPLYGNEGCGWAGWLLSPRGSRPTWGPTDPRHRSRNWGKVIDTLTCGFADLMGYIPVIGAPVGGVARALAHGVRVLEDGVNYATGNLPGCSFSIFLLALLSCVTVPVSAVEVRNISSSYYATNDCSDNSITWQRLVFEVTKWLLAILGSAHLLKASLLRVPYFVRAHALLRVCTLVRHLAGAKYIQMLLITVGRR

SEQ ID NO: 11 - DNA, 1161 bp, Hepatitis C virus
  CDS (229)..(1161)
ccaggcccccccctcccgggagagccatagtggtctgcggaaccggtgagtacaccggaattaccggaaagtctgggtcctttcttggataaacccactctatgtccggtcatttgggcgtgcccccgcaagactgctagccgagtagcgttgggttgcgaaaggccttgtggtactgcctgatagggtgcttgcgagtgccccgggaggtctcgtagaccgtgcatcatgagcacaaatcctaaacctcaaagaaaaaccaaaagaaacacaaaccgccgcccacaggacgtcaagttcccgggtggcggtcagatcgttggcggagtttacttgctgccgcgcaggggccccaggttgggtgtgcgcgcgacaaggaagacttccgagcgatcccagccgcgtgggagacgccagcccatcccgaaagatcggcgctccaccggcaagtcctggggaaagccaggatatccttggcccctgtacggaaacgagggttgcggctgggcaggttggctcctgtccccccgcgggtctcgtcctacttggggccccaccgacccccggcacagatcacgcaatttgggtaaagtcatcgatacccttacgtgtggttttgccgacctcatggggtacatccctgtcattggcgccccggtcggaggcgttgccagagccctagcgcacggtgttagggtcctggaagacggggtgaattacgcaacagggaatctacccggttgctctttttctatcttcttgcttgcccttctgtcgtgcgtcacagtgccagtgtctgcagtggaggtcaggaacattagttctagctactatgccactgacgattgctcgaacaacagcatcacctggcagcgccttgtgtttgaagtcacaaaatggctgttagcaatcctggggtctgcccacctccttaaagcgtccctgctacgggtgccatactttgtgagggctcacgctctgctacgggtgtgtaccctggtgaggcaccttgcaggagctaagtacatccagatgctgttgatcactgtaggcaggtggaccggcacttacatctatgtccacctctcccccttatcaacttgggcagctcagggtttgcgggacctggcggtcgccgtggagcctgtggtgttcagccca

SEQ ID NO: 12 - protein, 311 aa, Hepatitis C virus
MSTNPKPQRKTKRNTNRRPQDVKFPGGGQIVGGVYLLPRRGPRLGVRATRKTSERSQPRGRRQPIPKDRRSTGKSWGKPGYPWPLYGNEGCGWAGWLLSPRGSRPTWGPTDPRHRSRNLGKVIDTLTCGFADLMGYIPVIGAPVGGVARALAHGVRVLEDGVNYATGNLPGCSFSIFLLALLSCVTVPVSAVEVRNISSSYYATDDCSNNSITWQRLVFEVTKWLLAILGSAHLLKASLLRVPYFVRAHALLRVCTLVRHLAGAKYIQMLLITVGRWTGTYIYVHLSPLSTWAAQGLRDLAVAVEPVVFSP

SEQ ID NO: 13 - DNA, 1095 bp, Hepatitis C virus
  CDS (229)..(1095)
ccaggtccccccctcccgggagagccatagtggtctgcggaaccggtgagtacaccggaattgccaggacgaccgggtcctttcttggatcaacccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcatcatgagcacaaatcctaaacctcaaagaaaaaccaaacgtaacaccaaccgccgcccacaggacgtcaagttcccgggcggtggtcagatcgttggtggagtttacctgttgccgcgcaggggccccaggttgggtgtgcgcgcgactaggaagacttccgagcggtcgcaacctcgtggaaggcgacaacctatccccaaggctcgccagcccgagggtagggcctgggctcagcccgggtaccctcggcctagttggggccccacggacccccggcgtaggtcgcgtaatttgggtaaggtcatcgatacccttacatgcggcttcgccgacctcatggggtacatcccgctcgtcggcgcccccctagggggcgctgccagggccttggcgcatggcgtccgggttctggaggacggcgtgaactatgcaacagggaaccttcccggttgctctttctctatcttcctcttggctttgctgtcctgtttgaccattccagcctccgcccatgtcccccctctcaacgtccggggaggccgcgacgccatcatccttctcacatgtgcggtccactcagagctagtttttaaaatcaccaaaatcctgcttgcaatacttggtccgctcatggtgctccaggctggtctcattagggtgccgtacttcgtgcgcgcccaagggcttatccgtgcatgcatgttggtgcggaagatcgctgggggtcattatgtccaaatggctctcgtgaagctggccgcactgacgggcacgtacgtctatgaccatcttactccactgcgggactgggcccacacgggcctgcgagacctcgcggtggcggtcgagcccgtcgtcttctctgac

SEQ ID NO: 14 - protein, 289 aa, Hepatitis C virus
MSTNPKPQRKTKRNTNRRPQDVKFPGGGQIVGGVYLLPRRGPRLGVRATRKTSERSQPRGRRQPIPKARQPEGRAWAQPGYPRPSWGPTDPRRRSRNLGKVIDTLTCGFADLMGYIPLVGAPLGGAARALAHGVRVLEDGVNYATGNLPGCSFSIFLLALLSCLTIPASAHVPPLNVRGGRDAIILLTCAVHSELVFKITKILLAILGPLMVLQAGLIRVPYFVRAQGLIRACMLVRKIAGGHYVQMALVKLAALTGTYVYDHLTPLRDWAHTGLRDLAVAVEPVVFSD

SEQ ID NO: 15 - DNA, 788 bp, Hepatitis C virus
  CDS (1)..(786)
ctcttggctctgctgtcttgtctgaccatcctagcttccgcctatgaagtgcgcaacgtgtccgggttgtaccatgtcacgaacgactgctccaactcaagtattgtgtatgaggcagcggacatgatcatgcatacccccgggtgcgtgccctgcgtccgggagaacaaccgctctcgctgctgggtagcgctcacccctacgctcgcggccagaaacagcagcatccccactgcgacaatacgacgccatgtcgatttgctcgttggggcagccgctctctgctccgccatgtatgtgggggatctctgcggatctgtcttcctcgtgttcttctgtgctgcctggtatatcaagggtaagctggtccccggggcggcatatgctttttatagcgtatggccgctgctcctgctcttgctggcgctaccaccacgagcgtacgctatggaccgggagatggctgcatcatgtggaggcggggtcttcataggtctaataatcttgactttgtcaccgcactataaagcattcctcgctaggcttatatggtggttacaatattttatcaccaggaccgaggcgcacttgcaagtgtggatcccccctctcaacgttcgggggggccgtgatgccatcatcctcctcatgtgcgtggtccatccagagctaatttttgaaatcaccaagatcttgctcgccatactgggtccgcccatggtgctccaggccggcctgattagggtgccgtacttcgtgcgcgctcaagggctcattcgtgcatgcat

SEQ ID NO: 16 - protein, 262 aa, Hepatitis C virus
LLALLSCLTILASAYEVRNVSGLYHVTNDCSNSSIVYEAADMIMHTPGCVPCVRENNRSRCWVALTPTLAARNSSIPTATIRRHVDLLVGAAALCSAMYVGDLCGSVFLVFFCAAWYIKGKLVPGAAYAFYSVWPLLLLLLALPPRAYAMDREMAASCGGGVFIGLIILTLSPHYKAFLARLIWWLQYFITRTEAHLQVWIPPLNVRGGRDAIILLMCVVHPELIFEITKILLAILGPPMVLQAGLIRVPYFVRAQGLIRAC

SEQ ID NO: 17 - DNA, 1504 bp, Hepatitis C virus
  CDS (233)..(1504)
ccaggtccccccctcccgggagagccatagtggtctgcggaaccggtgagtacaccggaattgccaggacgaccgggtcctttcttggattaacccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcaccatggatgaataccaccgggttcaccaagacgtgcgggggccccccgtgtaacatcgggggggtcggcaataacaccctgacctgtcccacggactgcttccggaagcaccccgaggctacgtacacgcgatgcggttcggggccttggttgacacctaggtgcatggttgattacccatacaggctttggcactacccctgcactgtcaactttaccatcttcaaggttaggatgtacgtggggggcgtggagcacaggcttagtgctgcatgcaactggactcgaggagagcgttgtgacttggaggacagggatagatcagagctcagtccgctattgctatccacgacagagtggcaaatactgccctgctccttcaccaccctaccggctctgtccactggtttaatccatctccatcagaacatcgtggacgtgcaatacctgtacggtgtagggtcggcggttgtctcctttgtaatcaagtgggagtatgtcgtgctgcttttccttctcctggcggacgcgcgtgtctgtgcctgcttgtggatgatgctgctaatagcccaggctgaggccgccttagagaacctggtggtcctcaatgcggcgtccatagtcggaacgcatggcattctctccctccttgtattcttctgtgccgcctggtacatcaagggcaggctggtccctggggcggcatatgttctttatggtgtatggccgctgctccggctcctgctggcgttaccacaacgagcttacgccatggaccgggagatggctgcatcatgcggaggcgcggtttttataggcttggcactcttgaccttgtcaccatactacaaagtgttcctcgctaggctcatatggtggttacaatactttatcactagagccgaggcgcacttgcaagtgtgggtccccccccttaacgctcggggaggccgcgatgccatcatccttctcacatgtgcagtccatccagagctaatctttgacatcaccaaaatcctgctcgccatatttggtccactcatggtcctccaggctagtataactgcagtgccgtactttgtgcgcgctcaagggctcattcgtgcatgcatgttggtgcggaaagttgctgggggccattatgtccaaatggccttcatgaagctggcagcactgacaggtacgtacgtttacgaccatcttactccgctgcgggactgggcccacgcgggcctacgggaccttgcggtggcagtagagcccgttgtcttctctgac

SEQ ID NO: 18 - protein, 424 aa, Hepatitis C virus
MNTTGFTKTCGGPPCNIGGVGNNTLTCPTDCFRKHPEATYTRCGSGPWLTPRCMVDYPYRLWHYPCTVNFTIFKVRMYVGGVEHRLSAACNWTRGERCDLEDRDRSELSPLLLSTTEWQILPCSFTTLPALSTGLIHLHQNIVDVQYLYGVGSAVVSFVIKWEYVVLLFLLLADARVCACLWMMLLIAQAEAALENLVVLNAASIVGTHGILSLLVFFCAAWYIKGRLVPGAAYVLYGVWPLLRLLLALPQRAYAMDREMAASCGGAVFIGLALLTLSPYYKVFLARLIWWLQYFITRAEAHLQVWVPPLNARGGRDAIILLTCAVHPELIFDITKILLAIFGPLMVLQASITAVPYFVRAQGLIRACMLVRKVAGGHYVQMAFMKLAALTGTYVYDHLTPLRDWAHAGLRDLAVAVEPVVFSD

SEQ ID NO: 19 - DNA, 245 bp, Hepatitis C virus
  CDS (1)..(243)
ctcttggctttgttgtcctgtttgaccgtcccgacttccgcttatgaagtgcgcaacgtgtccgggatgtaccaagtcacgaacgactgctccaactcaagcattgtgtatgaggcagcgggcgcgatcatctttgagattaccaaaatcttgctcgccatgcttggtccgctcatgatgctccaggctggcctaattagagtgccgtacttcgtgcgcgctcaagggctcattcgtgcgtgctc

SEQ ID NO: 20 - protein, 81 aa, Hepatitis C virus
LLALLSCLTVPTSAYEVRNVSGMYQVTNDCSNSSIVYEAAGAIIFEITKILLAMLGPLMMLQAGLIRVPYFVRAQGLIRAC

SEQ ID NO: 21 - DNA, 1119 bp, Hepatitis C virus
  CDS (229)..(1119)
ccaggaccccccctcccgggagagccatagtggtctgcggaaccggtgagtacaccggaattgccaggacgaccgggtcctttcttggattaacccgctcaatgcccggagatttgggcgtgcccccgcaagactgctagccgagtagtgttgggtcgcgaagggccttgtggtactgcctgatagggtgcttgcgagtgccccgggaggtctcgtagaccgtgcaccatgagcacgaatcctaaaccccaaagaaaaaccaaccgaaacaccaaccgccgtccacaggacgttaagttcccgggcggtggtcagatcgtcggtggagtttacctgttgccgcgcaggggccccaggttgggtgtgcgcgcgactaggaagacttccgagcggtcgcaacctcgtggaaggcgacaacctatccccaaggttcgccggcccgagggcaggacctgggctcagcccgggtatccttggcccctctatggcaatgagggcttggggtgggcaggatggctcctgtcaccccgtggctaccggcctagttggggccccacggacccccggcgtaggtcgcgtaatttgggtaaggtcatcgataccctcacatgcggcttcgccgacctcatggggtatattccgcttgtcggcgcccctttaggaggcgctgccagggccctggcacatggtgtccgggttctggaggacggcgtgaattatgcaacagggaatttgcctggttgctctttctctatcttcctcttggctctgctgtcctgttttaccaccccagcttccgcttatggagtgcgcacgtgcgcggtccatccagagccaatctttgacatcaccaacctcctgctcgccatactcggcccgctcatggtgctccaggctggcataactagagtgccgtacttcgtacgcgctcaggggctcattcgtgcatgcatgttagtgaggaaagcgcctgggggtcattatgtccaaatggccctcatgaggctggccgcgctgacaggtacgtacgtgtatgaccatctcgccccattgcagcattgggcccacgcgggcctacgagaccttgcggtggcagtagaacccgtcatcttctctgac

SEQ ID NO: 22 - protein, 297 aa, Hepatitis C virus
MSTNPKPQRKTNRNTNRRPQDVKFPGGGQIVGGVYLLPRRGPRLGVRATRKTSERSQPRGRRQPIPKVRRPEGRTWAQPGYPWPLYGNEGLGWAGWLLSPRGYRPSWGPTDPRRRSRNLGKVIDTLTCGFADLMGYIPLVGAPLGGAARALAHGVRVLEDGVNYATGNLPGCSFSIFLLALLSCFTTPASAYGVRTCAVHPEPIFDITNLLLAILGPLMVLQAGITRVPYFVRAQGLIRACMLVRKAPGGHYVQMALMRLAALTGTYVYDHLAPLQHWAHAGLRDLAVAVEPVIFSD

SEQ ID NO: 23 - DNA, 1143 bp, Hepatitis C virus
  CDS (229)..(1143)
ccaggtccccccctcccgggagagccatagtggtctgcggaaccggtgagtacaccggaattgccaggacgaccgggtcctttcttggatcaacccgctcaatgcctggagatttgggcgtgcccccgcgagactactagccgagtagtgttgggtcgcgaaaggccttgtggtactgcctgatagggtgcttgcgagtgccccgggaggtctcgtagaccgtgcatcatgagcacaaatcctaaacctcaaagaaaaaccaaacgtaacaccaaccgccgcccacaggacgtcaagttcccgggcggtggccagatcgttggtggagtttacctgttgccgcgcaggggccccaggttgggtgtgcgcgcgactaggaagacttccgagcggtcgcaacctcgtggaaggcgacaacctatccccaaggctcgccgacccgagggcagggcctgggcacagcccgggtacccttggcctctctatggcaatgagggcctggggtgggctggatggctcctgtcaccccgcggctcccggcctagttggggccccacggacccccggcgtaggtcgcgcaatttgggtaaggtcatcgataccctcacttgcggcttcgccgacctcatggggtacattccgctcgtcggcgcccccctaggaggtgctgccagggccctggcgcatggcgtccgggttctggaagacggcgtgaactacgcaacagggaatttgcccggttgctctttctctatcttcctcttggctttactgtcctgtttgaccatcccagcttccgctcatcaagtgcgcaacgtgtccggggtgtaccatgtcacgaacaactgctccaactcaagaattgtggacatcaccaagattttgctcgccatatttggcccgctcatggcgctccaggctggtttaactagagtgccgtactttgtacgcgctcatgggctcatccgtgtgtgcatgttggtgcggaaagtctctgggggtcattacgtccagatggctctcatgaggctggccgcactgacgggcacgtacgtctataaccatcttactccgctgcgggactgggcccacgtgggcctgcgagaccttgcagtggcagttgagcctgtcatcttctctgac

SEQ ID NO: 24 - protein, 305 aa, Hepatitis C virus
MSTNPKPQRKTKRNTNRRPQDVKFPGGGQIVGGVYLLPRRGPRLGVRATRKTSERSQPRGRRQPIPKARRPEGRAWAQPGYPWPLYGNEGLGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTIPASAHQVRNVSGVYHVTNNCSNSRIVDITKILLAIFGPLMALQAGLTRVPYFVRAHGLIRVCMLVRKVSGGHYVQMALMRLAALTGTYVYNHLTPLRDWAHVGLRDLAVAVEPVIFSD

SEQ ID NO: 25 - DNA, 844 bp, Hepatitis C virus
  CDS (1)..(843)
cttttgactttactgtcctgtttgaccatcccagcttccgctcatcaagtgcgcaacgtgtccggggtgtaccatgtcatgaacaactgctccaactcaagtattgtggacatcaccaagattttgctcgccatatttggcccgctcatggtgctccaggctggtttaactagagtgccgtacttcgtacgcgctcatgggctcatccgtgtgtgcatgttggtgcggaaagtctctgggggtcattacgtccagatggctctcatgaggctggccgcactgacgggtacgtacgtctataaccatcttactccgctgcgggactggggccacgcgggcctgcgagaccttgcagtggcagttgagcctgtcatcttctctgacatggagaccaagatcatcacctggggggcggacaccgcggcgtgcggggacatcatctcaggtctacccgtctccgcccgaagggggagggagatattactgggaccggccgacagttttggagagcgagggtggcgactccttgcgcctattacggcctactcccaacaaacccggggcctgcttggctgcatcatcactagccttacaggtcgggacaagaaccaggttgagggggaggttcaggtggtttccaccgcaacgcaatctttcccggcgacctgcgtcaacggcgtacgttggactgtctaccatggtgccggctcaaagaccctagccggcccaaagggcccagtcacccagatgtacaccaatgtagaccgggacctcgttggttggccggcgccctctggggcgcgctccttgacaccatgcacctgtggcagctcagacctctacc

SEQ ID NO: 26 - protein, 281 aa, Hepatitis C virus
LLTLLSCLTIPASAHQVRNVSGVYHVMNNCSNSSIVDITKILLAIFGPLMVLQAGLTRVPYFVRAHGLIRVCMLVRKVSGGHYVQMALMRLAALTGTYVYNHLTPLRDWGHAGLRDLAVAVEPVIFSDMETKIITWGADTAACGDIISGLPVSARRGREILLGPADSFGERGWRLLAPITAYSQQTRGLLGCIITSLTGRDKNQVEGEVQVVSTATQSFPATCVNGVRWTVYHGAGSKTLAGPKGPVTQMYTNVDRDLVGWPAPSGARSLTPCTCGSSDLY

SEQ ID NO: 27 - DNA, 1042 bp, Hepatitis C virus
  CDS (229)..(1041)
ccaggtccccccctcccgggagagccatagtggtctgcggaaccggtgagtacaccggaattgccaggacgaccgggtcctttcttggatcaacccgctcaatgcctggagatttgggcgtgcccccgcgagactactagccgagtagtgttgggtcgcgaaaggccttgtggtactgcctgatagggtgcttgcgagtgccccgggaggtctcgtagaccgtgcatcatgagcacaaatcctaaacctcaaagaaaaaccaaacgtaacaccaaccgccgcccacaggacgtcaagttcccgggcggtggccagatcgttggtggagtttacctgttgccgcgcaggggccccaggttgggtgtgcgcgcgactaggaagacttccgagcggtcgcaacctcgtggaaggcgacaacctatccccaaggctcgccgacccgagggcagggcctgggcacagcccgggtacccttggcctctctatggcaatgagggcctggggtgggcaggatggttcctgtcaccccgcggctcccggcctagttggggccccacggacccccggcgtaggtcgcgcaatttgggtaaggtcatcgataccctcacttgcggcttcgccgacctcatggggtacattccgctcgtcggcgcccccttaggaggtgctgccagggccctggcgcatggcgtccgggttctggaagacggcgtggactacgcaacagggaatttgcccggttgctctttctctatcttcctcttggctttactgtcctgtttgaccatcccagcttccgctcatcaagtgcgcgacgtgtccggggtgtaccatgtcacgaacaactgctccaactcaagtattgtggtcatcaccaagattctgctcgccatatttggcccgctcatggcgctccaggctggtttaactagagtgccgtacttcgtacgcgctcatgggctcatccgtgtatgcatgttggtgcggaaagtctctgggggtcattacgtccagatggctctcatgaggctggccgcactga

SEQ ID NO: 28 - protein, 271 aa, Hepatitis C virus
MSTNPKPQRKTKRNTNRRPQDVKFPGGGQIVGGVYLLPRRGPRLGVRATRKTSERSQPRGRRQPIPKARRPEGRAWAQPGYPWPLYGNEGLGWAGWFLSPRGSRPSWGPTDPRRRSRNLGKVIDTLTCGFADLMGYIPLVGAPLGGAARALAHGVRVLEDGVDYATGNLPGCSFSIFLLALLSCLTIPASAHQVRDVSGVYHVTNNCSNSSIVVITKILLAIFGPLMALQAGLTRVPYFVRAHGLIRVCMLVRKVSGGHYVQMALMRLAAL

SEQ ID NO: 29 - DNA, 232 bp, Hepatitis C virus
  CDS (1)..(231)
ctcttggctttgctgtcctgtttgaccattccagcctccgcccatgtcccccctctcaacgtccggggaggccgcgacgccatcatccttctcacatgtgcggtccactcagagctagtttttaaaatcaccaaaatcttgcttgcaatacttggtccgctcatggtgctccaggctggtctcattagggtgccgtacttcgtgcgcgcccaagggcttatccgtgcatgca

SEQ ID NO: 30 - protein, 77 aa, Hepatitis C virus
LLALLSCLTIPASAHVPPLNVRGGRDAIILLTCAVHSELVFKITKILLAILGPLMVLQAGLIRVPYFVRAQGLIRAC

SEQ ID NO: 31 - DNA, 1823 bp, Hepatitis C virus
  CDS (228)..(1823)
ccaggtcccccctcccgggagagccatagtggtctgcggaaccggtgagtacaccggaattgccaggacgaccgggtcctttcttggatcaacccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcatcatgagcacaaatcctaaacctcaaagaaaaaccaaacgtaacaccaaccgccgcccacaggacgtcaagttcccgggcggtggtcagatcgttggtggagtttacctgttgccgcgcaggggccccaggttgggtgtgcgcgcgactaggaagacttccgagcggtcgcaacctcgtggaaggcgacaacctatccccaaggctcgccagcccgagggtagggcctgggctcagcccgggtacccttggcccctctacggcaatgagggcctggggtgggcaggatggctcctgtcaccccgcggctctcggcctagttggggccccacagacccccggcgtaggtcgcgtaatttgggtaaggtcatcgatacccttacatgcggcttcgccgacctcacggggtacatcccgctcgtcggcgcccccctagggggcgctgccagggccttggcgcatggcgtccgggttctggaggacggcgtgaactatgcaacagggaaccttcccggttgctctttctctatcttcctcttggctttgctgtcctgtttgaccattccagcctccgcccatgtcccccctctcaacgtccggggaggccgcgacgccatcatccttctcacatgtgcggtccactcagagctagtttttaaaatcaccaaaatcttgcttgcaatacttggtccgctcatggtgctccaggctggtctcgttagggtgccgtacttcgtgcgcgcccaagggcttatccgtgcatgcatgttggtgcggaagatcgctgggggtcattatgtccaaatggctttcgtgaagctggccgcactgacgggcacgtacgtctatgaccatcttactccactgcgggactgggcccacacgggcctgcgagacctcgcggtggcggtcgagcccgtcgtcttctctgacatggggaccaagatcatcacctggggggcggacaccgcggcgtgcggggacatcatctcgggtctgcccgtctccgctcggagggggagggagatactcctgggactggccgatagtttcggagagcagggatggcgactccttgcgcctatcacggcctactcccaacagacgcggggtttacttggctgcatcatcactagcctcacaggccgggacaagaaccaggtcgagggggaagtccaggtggtttccaccgcaacgcagtctttcctcgcgacatgtgtaaatggtgtgtgttggactgtctaccatggtgccggctcaaagaccttagccggccctaagggtccgatcactcaaatgtacaccaatgtggaccaggacctcgttggctggcaggcgccccctggggcgcgttccatgacaccatgcacctgcggcagctcggacctctacctggtcacgagacatgccgatgtcattccggtgcgtcggcggggcgacagcagagggagcctactctcccccaggcctgtgtcctatttgaagggctcctcgggtggtccactgctctgccccttggggcacgtcgtgggcatcttccgggctgctgtgtgcacccggggggttgcgaaggcggtggactttgtacccgttgagtct

SEQ ID NO: 32 - protein, 532 aa, Hepatitis C virus
MSTNPKPQRKTKRNTNRRPQDVKFPGGGQIVGGVYLLPRRGPRLGVRATRKTSERSQPRGRRQPIPKARQPEGRAWAQPGYPWPLYGNEGLGWAGWLLSPRGSRPSWGPTDPRRRSRNLGKVIDTLTCGFADLTGYIPLVGAPLGGAARALAHGVRVLEDGVNYATGNLPGCSFSIFLLALLSCLTIPASAHVPPLNVRGGRDAIILLTCAVHSELVFKITKILLAILGPLMVLQAGLVRVPYFVRAQGLIRACMLVRKIAGGHYVQMAFVKLAALTGTYVYDHLTPLRDWAHTGLRDLAVAVEPVVFSDMGTKIITWGADTAACGDIISGLPVSARRGREILLGLADSFGEQGWRLLAPITAYSQQTRGLLGCIITSLTGRDKNQVEGEVQVVSTATQSFLATCVNGVCWTVYHGAGSKTLAGPKGPITQMYTNVDQDLVGWQAPPGARSMTPCTCGSSDLYLVTRHADVIPVRRRGDSRGSLLSPRPVSYLKGSSGGPLLCPLGHVVGIFRAAVCTRGVAKAVDFVPVES

SEQ ID NO: 33 - DNA, 1824 bp, Hepatitis C virus
  CDS (229)..(1824)
ccaggtccccccctcccgggagagccatagtggtctgcggaaccggtgagtacaccggaattgccaggacgaccgggtcctttcttggatcaacccgctcaatgcctggagatttgggcgtgcccccgcgaggctgctagccgagtagtgttgggtcgcgaaaggccttgtggtactgcctgatagggtgcttgcgagtgccccgggaggtctcgtagaccgtgcatcatgagcacaaatcctaaacctcaaagaaaaaccaaacgtaacaccaaccgccgcccacaggacgtcaatttcccgggcggtggtcagatcgttggtggagtttacctgttgccgcgcaggggccccaggttgggtgtgcgcgcgactaggaagacttccgagcggtcgcaacctcgtggaaggcgacaacctatccccaaggctcgccagcccgagggtagggcctgggctcagcccgggtacccttggcccctctacggcaatgagggcctggggtggacaggatggctcctgtcaccccgcggctctcggcctagttggggccccacggacccccggcgtaggtcgcgtaatttgggtaaggtcatcgatacccttacatgcggcttcgccgacctcatggggtacatcccgctcgtcggcgcccccctagggggcgctgccagggccttggcgcatggcgtccgggttctggaggacggcgtgaactatgcaacagggaaccttcccggttgctctttctctatcttcctcttggctttgctgtcctgtttgaccattccagcctccgcccatgtcccccctctcaacgtccggggaggccgcgacgccatcatccttctcacatgtgcggtccactcagagctagtttttaaaatcaccaaaatcttgcttgcaatacttggtccgctcatggtgctccaggctggtctcattagggtgccgtacttcgtgcgcgcccaagggcttatccgtgcatgcatgttggtgcggaagatcgctgggggtcattatgtccaaatggctttcgtgaagctggccgcactgacgggcacgtacgtctatgaccatcttactccactgcgggactgggcccacacgggcctgcgagacctcgcggtggcggtcgagcccgtcgtcttctctgacatggagaccaagatcatcacctggggggcggacaccgcggcgtgcggggacatcatctcgggtctgcccgtctccgctcggagggggagggagatactcctgggacgggccgatagtttcggagagcagggatggcgactccttgcgcctatcacggcctactcccaacagacgcggggtttacttggctgcatcatcactagcctcacaggccgggacaagaaccaggtcgagggggaagtccaggtggtttccaccgcaacgcagtctttcctcgcgacatgtgtaaatggtgtgtgttggactgtctaccatggtgccggctcaaagaccttagccggccctaagggtccgatcactcaaatgtacaccaatgtggaccaggacctcgttggttggcaggcgccccctggggcgcgttccatgacaccatgcacctgcggcagctcggacctctacctggtcacgagacatgccgatgtcattccggtgcgtcggcggggcgacagcagagggagcctactctcccccaggcctgtgtcctatttgaagggctcctcgggtggtccactgctctgccccttggggcacgtcgtgggcatcttccgggctgctgtgtgcacccggggggttgcgaaggcggtggactttgtacccgttgagtct

SEQ ID NO: 34 - protein, 532 aa, Hepatitis C virus
MSTNPKPQRKTKRNTNRRPQDVNFPGGGQIVGGVYLLPRRGPRLGVRATRKTSERSQPRGRRQPIPKARQPEGRAWAQPGYPWPLYGNEGLGWTGWLLSPRGSRPSWGPTDPRRRSRNLGKVIDTLTCGFADLMGYIPLVGAPLGGAARALAHGVRVLEDGVNYATGNLPGCSFSIFLLALLSCLTIPASAHVPPLNVRGGRDAIILLTCAVHSELVFKITKILLAILGPLMVLQAGLIRVPYFVRAQGLIRACMLVRKIAGGHYVQMAFVKLAALTGTYVYDHLTPLRDWAHTGLRDLAVAVEPVVFSDMETKIITWGADTAACGDIISGLPVSARRGREILLGRADSFGEQGWRLLAPITAYSQQTRGLLGCIITSLTGRDKNQVEGEVQVVSTATQSFLATCVNGVCWTVYHGAGSKTLAGPKGPITQMYTNVDQDLVGWQAPPGARSMTPCTCGSSDLYLVTRHADVIPVRRRGDSRGSLLSPRPVSYLKGSSGGPLLCPLGHVVGIFRAAVCTRGVAKAVDFVPVES

SEQ ID NO: 35 - DNA, 1115 bp, Hepatitis C virus
  CDS (229)..(1113)
ccaggaccccccctcccgggagagccatagtggtctgcggaaccggtgagtacaccggaattgccaggacgaccgggtcctttcttggatcaacccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcaccatgagcacgaatcctaaacctcaaaaaaaacccaaatgtaacaccaaccgccgcccacaggacgtcaagttcccgggcggtggtcagatcgttggtggagtttacctgttgccgcgcaggggccccaggttgggtgtgcgcgcgactaggaagacttccgagcggtcgcaacctcgtggaaggcgacaacctatccccaaggctcgccggcccgagggtagggcctgggctcagcccgggtacccttggcccctctatggcgatgagggcctagggtgggcaggatggctcctgtcaccccgcggctcccggcctagttggggccccactgacccccggcgtaggtcgcgtaatctgggtaaggtcatcgataccctcacatgcggcttcgccgacctcatggggtacattccgctcgtcggcgcccccttaggaggcgttgccagggccctggcgcatggcgtccgggttctggaagacagcgtgaactacgcaacagggaatctgcccggttgctctttctctatcttcctcttagctttgctgtcctgcttgactgtcccggcttccgcttgcaaaactcccacgctcgcggccagggagctaaaccttggaatcgccacaatcttgctcgccatatttggtccgctcgtggcgctccagactggcctatttagggtgccgtacttcgtgcgcgcccaagggctcatccgtgcgtgcatgttggtgcggaaagtctctgggggtcatcatgtccaaatggctcttgtgaggctagctgctctaacgggcacgtacgtttatgaccatcttactccgctgcgggactggcccacgcgggcctgcgagatcttgcggtggctgttgagcccgtcatcttctctgac

SEQ ID NO: 36 - protein, 295 aa, Hepatitis C virus
MSTNPKPQKKPKCNTNRRPQDVKFPGGGQIVGGVYLLPRRGPRLGVRATRKTSERSQPRGRRQPIPKARRPEGRAWAQPGYPWPLYGDEGLGWAGWLLSPRGSRPSWGPTDPRRRSRNLGKVIDTLTCGFADLMGYIPLVGAPLGGVARALAHGVRVLEDSVNYATGNLPGCSFSIFLLALLSCLTVPASACKTPTLAARELNLGIATILLAIFGPLVALQTGLFRVPYFVRAQGLIRACMLVRKVSGGHHVQMALVRLAALTGTYVYDHLTPLRDWPTRACEILRWLLSPSSSL

SEQ ID NO: 37 - DNA, 817 bp, Hepatitis C virus
  CDS (1)..(816)
ctcttagctttgctgtcctgcttgactgtcccagcttccgcttgcgaaactcccacgctcgcggccagggagctaaaccttggaatcgccaaaatcttgctcgccatatttggtccgctcatggtgctccagactggcctaattagggtgccgtacttcgtgcgcgcccaggggctcatccgtgcgtgcatgttggtgcggaaagtctctgggggtcattatgtccaaatggctcttgtgaggctagctgcgctaacgggcacgtacgtttatgaccatcttactccgctgcgggactgggcccacgcgggcctgcgagatctcgcggtggcagttgagcccgtcatcttctctgacatggagaccaagatcatcacctgggaggcagacaccgcggcgtgcggggacatcatctcgggcctacccgtctccgcccgaagggggagggagatacttttggggccggccgatagttttagagatcaggggtggcaactccttgcgcccatcacggcctactcccaacagacgcggggcctacttggctgcatcatcactagcctcacaggccgggacaagaaccaggtcgagggagaggctcaggtggtttccaccgcaacacaatccttcctggcgacctgtgttaatggcgtgtgttggaccgcctaccgtggcgccggtgcaaagaccctagccggcccaaagggtccaatcacccaaatgtataccaatgtagaccaggacctcgtcggttggcaggcgccctccgggtcgcgttccttaacgccatgcacctgcggtagctcggacctttact

SEQ ID NO: 38 - protein, 272 aa, Hepatitis C virus
LLALLSCLTVPASACETPTLAARELNLGIAKILLAIFGPLMVLQTGLIRVPYFVRAQGLIRACMLVRKVSGGHYVQMALVRLAALTGTYVYDHLTPLRDWAHAGLRDLAVAVEPVIFSDMETKIITWEADTAACGDIISGLPVSARRGREILLGPADSFRDQGWQLLAPITAYSQQTRGLLGCIITSLTGRDKNQVEGEAQVVSTATQSFLATCVNGVCWTAYRGAGAKTLAGPKGPITQMYTNVDQDLVGWQAPSGSRSLTPCTCGSSDLY

SEQ ID NO: 39 - DNA, 2302 bp, Hepatitis C virus
  CDS (1)..(2301)
cctttggctctgctatcctgtctgactgtcccggcttccgcttatgaggtgcgcaacttctccgggatataccgtgtcacgaacgactgctccaactcaagcattgtgtatgaggcagcggacatgatcatgcatactcccgggtgtgtgccctgcgttcgggagggtaactcctcccgttgctgggtagcgctcactcccacgctagcggccaggaatatcagcgtccccactacgacaatacgacgcaatgtcgacttgctcgttggggcggctgctttctgctccgccatgtacgtgggagacctctgcggatctgttttcctcgtctcccagcttttcaccttctcgcctcgccggcatgagacagtacaggactgcaattgttcaatctattccggccatgtgtcaggtcaccgtatggcttgggatatgatgatgaactggtcacctacagcaaccctagtagtgtcacagttactccggatcccacaagccgtcgtggacatggtggcgggggctcactggggagtcctagcgggccttgcctactatcccatggcgggaaactgggctaaggtgttaattgtattgctgctcttcgccggcgttgacgggcagacccgcgtgacagggggggcggcagctcacaccgcccgtgggctcacttccatccttccacctgggccgtctcagaacatccagcttgtaaacaccaatggcagctggcacatcaacaggactgctctgaactgcaatgacagcctccagactgggtttcttgccgcgctgttcttcacacacaagttcaacgcgtctggatgcccagaacgcatggccagctgccgtaccattgacaagttcaatcaagggtggggtcccatcacctatgatgggcatggcggccaggaccagaggccttattgctggcactacgcgcctaagccgtgcggtatcgtacccgcgtcgcaggtgtgtggtccagtgtattgtttcaccccaagcccagttgtggtggggacgaccgatcgctccggtgtccctacgtatagctggggggagaatgagacagacgtgctgcttcttaacaacacgcggccgccgctaggcaactggttcggctgtacatggatgaattgcactgggttcaccaagacgtgcgggggccccccgtgtaatatcgggggagtcggcaacaacaccttgacctgccccacggattgcttccggaagcaccccgaagccacttacaccaaatgtggttcggggccttggttgacacccagatgcatagttgactacccatacaggctctggcactacccctgcactgtcaacttcaccatcttcaaggttaggatgtatgtggggggcgtggagcacaggctcaatgctgcatgcaattggacccgaggggagcgttgtgggttggaggacagggatagatcggagctcagcccgctgctgctatctacaacagagtggcagatactgccttgctccttcaccacactaccggctctgtccactggtttaatccatcttcaccagaacatcgtggacgtgcaatatctgtacggcatagggtcggtggttgtctcctctgcaatcaagtgggagtatgtcgtgttgctcttccttctcctggcggacgcacgcgtctgtgcctgcttgtggatgatgctactggtagcccaggccgaggctgctttagagaacctagtggttctcaacgcggcatccgtggctgggacgcacggcattatccccttccttgtgttcttctgtgccgcctggtacatcaaaggcaggctcgtccctgcagcggcatatgctttctatggcgtatggccgctgctcctgctcctgctggcgttaccaccacgagcttacgccatggaccgggagatggctgcatcgtgtggaggcggggtttttgtaggtctggcattcttgaccttgtcaccatactacaaggtgttcctcgctaagctcatatggtggttacaatattttatcaccagagccgaggcgcacttgcaagtgtggatcccccccctcaacgttcggggaggccgtgatgccatcatcctcctcgcatgcgcagtccacccggagctaatctttgacatcaccaaacttctgctcgccatactcggcccgctcatggtgttccaggccagcataacccgagtgccgtactttgtgcgcgctcaagggctcattcgtgcatgcatgttagtgcggaaagccgctgggggtcattatatccaaatggccctcgtgaaactggccgcgctga

SEQ ID NO: 40 - protein, 767 aa, Hepatitis C virus
PLALLSCLTVPASAYEVRNFSGIYRVTNDCSNSSIVYEAADMIMHTPGCVPCVREGNSSRCWVALTPTLAARNISVPTTTIRRNVDLLVGAAAFCSAMYVGDLCGSVFLVSQLFTFSPRRHETVQDCNCSIYSGHVSGHRMAWDMMMNWSPTATLVVSQLLRIPQAVVDMVAGAHWGVLAGLAYYPMAGNWAKVLIVLLLFAGVDGQTRVTGGAAAHTARGLTSILPPGPSQNIQLVNTNGSWHINRTALNCNDSLQTGFLAALFFTHKFNASGCPERMASCRTIDKFNQGWGPITYDGHGGQDQRPYCWHYAPKPCGIVPASQVCGPVYCFTPSPVVVGTTDRSGVPTYSWGENETDVLLLNNTRPPLGNWFGCTWMNCTGFTKTCGGPPCNIGGVGNNTLTCPTDCFRKHPEATYTKCGSGPWLTPRCIVDYPYRLWHYPCTVNFTIFKVRMYVGGVEHRLNAACNWTRGERCGLEDRDRSELSPLLLSTTEWQILPCSFTTLPALSTGLIHLHQNIVDVQYLYGIGSVVVSSAIKWEYVVLLFLLLADARVCACLWMMLLVAQAEAALENLVVLNAASVAGTHGIIPFLVFFCAAWYIKGRLVPAAAYAFYGVWPLLLLLLALPPRAYAMDREMAASCGGGVFVGLAFLTLSPYYKVFLAKLIWWLQYFITRAEAHLQVWIPPLNVRGGRDAIILLACAVHPELIFDITKLLLAILGPLMVFQASITRVPYFVRAQGLIRACMLVRKAAGGHYIQMALVKLAAL

SEQ ID NO: 41 - DNA, 2240 bp, Hepatitis C virus
  CDS (1)..(2238)
ctcttggctctgctgtcctgtctgactatcccagcttccgcctatgaggtgcgcaacgtgtccgggttgtaccatgtcacgaacgactgctccaactcaagtattgtgtatgaggcagcggacatgatcatgcatacccccgggtgcgtgccctgcgtccgggagaacaaccgccctcgctgctgggtagcgctcactcccacgctcgcggccagaaacagcagcatccccactgcgacaatacgacgccatgtcgatttgctcgttggggcagccgctctctgctccgccatgtatgtgggggatctttgcggatctgtcttcctcgtctcccagctgttcaccttctcgcctcgccggtatgagacggtacaagactgcaattgctcactctatcctggccacgtaacaggtcaccgcatggcctgggatatgatgatgaactggtcgcctacaacagccctagtggtatcgcagatactgcggatcccacaagccgtcatggacatggtgacgggggcccactggggagtcctggcgggcctcgcctactattccatggtgggaaactgggctaaggtcctgattgtgttgttactctttgccggcgttgatgggaccacccacataacgggggggactgcaggccaaactgcctttagcctcacaagtctcctcgcatctgggccgactcagaagatccaaattataaacactaacggcagctggcacatcaacagaactgccttgagttgtaacgactcccttcagactgggttcattgccgcgctgttctacaagcacaggttcaactcgtccggatgcccagagcgcatggccagctgccgccccatcgacaggtttaatcaggggtggggtcccattacttatgatgataagcttcccgtctcagaccagaggccttactgcaggcactacgcgcctcggccgtgcggtatcgtgcccgcgtcggaggtgtgtggtccggtgtattgcttcaccccaagccctgttgtggtggggacgaccgatcgcttcggcgctcccacgtataactggggggagaatgagacggacgtgctaatcctcaacaacacgcggccgccgcaaggcaactggtttggctgtacatggatgaataacaccgggttcaccaagacgtgcggaggccctccgtgtaacatcgggggggtcggcaatgagaccttgacctgccctacggattgcttccggaagcaccccgaagccacgtacaccaaatgcggctcggggccttggttgacacctaggtgtatggttgattacccatacagactttggcactacccctgcactgtaaactttactatcttcaagatcaggatgtacgtggggggtgtagagcacaggttcacagccgcgtgcaattgggcccgaggagagcgctgtgacgtagaggacagggatagagcagagctcagcccgctactactgtctacaactgagtggcagatactgccctgttcctttaccaccctaccagctctgtccaccggatcgatccacctccatcagaacaccgtggacgtgcaatacctgtacggtgtagggtcagcggttgtttccatcgcgatcaaatgggagtatgtcctgctgcttttccttctcttggcggacgcacgcgtctgcgcctgtttgtggatgatgctgctgatagcccaggctgaggccgctttggagaacctggtgatcctcaatgcggcgtccgtagctggagcgcacggcattctttccttcctcatgttcttctgtgctgcctggtatatcaagggtaagctggtccccggggcggcatatgctttctatagcgtatggccgctgctcctgctcctgctggcgctaccaccacgagcgtacgctatggaccgggagatggctgcatcatgtgggggtggggtcttcataggtttggtagtcttgactttgtcaccgcactataaagcattcctcgctaggcttatatggtggttacaatattttatcaccaggaccgaggcgcacttgcaagtgtggatcccccccctcaacgttcgggggggccgtgatgccatcatcctcctcatgtgcgtggtccatccagagctaatttttgaaatcaccaagatcttgctcgccatactgggtccgcccatggtgctccaggccggcctgattagggtgccgtacttcgtgcgcgctcaagggctcattcgtgcgtgcat

SEQ ID NO: 42 - protein, 746 aa, Hepatitis C virus
LLALLSCLTIPASAYEVRNVSGLYHVTNDCSNSSIVYEAADMIMHTPGCVPCVRENNRPRCWVALTPTLAARNSSIPTATIRRHVDLLVGAAALCSAMYVGDLCGSVFLVSQLFTFSPRRYETVQDCNCSLYPGHVTGHRMAWDMMMNWSPTTALVVSQILRIPQAVMDMVTGAHWGVLAGLAYYSMVGNWAKVLIVLLLFAGVDGTTHITGGTAGQTAFSLTSLLASGPTQKIQIINTNGSWHINRTALSCNDSLQTGFIAALFYKHRFNSSGCPERMASCRPIDRFNQGWGPITYDDKLPVSDQRPYCRHYAPRPCGIVPASEVCGPVYCFTPSPVVVGTTDRFGAPTYNWGENETDVLILNNTRPPQGNWFGCTWMNNTGFTKTCGGPPCNIGGVGNETLTCPTDCFRKHPEATYTKCGSGPWLTPRCMVDYPYRLWHYPCTVNFTIFKIRMYVGGVEHRFTAACNWARGERCDVEDRDRAELSPLLLSTTEWQILPCSFTTLPALSTGSIHLHQNTVDVQYLYGVGSAVVSIAIKWEYVLLLFLLLADARVCACLWMMLLIAQAEAALENLVILNAASVAGAHGILSFLMFFCAAWYIKGKLVPGAAYAFYSVWPLLLLLLALPPRAYAMDREMAASCGGGVFIGLVVLTLSPHYKAFLARLIWWLQYFITRTEAHLQVWIPPLNVRGGRDAIILLMCVVHPELIFEITKILLAILGPPMVLQAGLIRVPYFVRAQGLIRAC

SEQ ID NO: 43 - DNA, 2237 bp, Hepatitis C virus
  CDS (1)..(2226)
ctcttggctttgttgtcctgtttgaccgtcccaacttccgcttatgaagtgcgcaacgtgtccgggatgtaccaagtcacgaacgactgctccaactcaagcattgtgtatgaggcagcggacgtgatcatgcacacccccgggtgcgtgccctgtgtccgggagagcaacctctcccgctgctgggttgcgctcacgcccacgctcgcggccaggaacagcagtatccccactacgacaatacgacgtcatgtcgatttgctcgttggggcatctgccttctgctccgctatgtacgtgggggatctttgcggatctgtcttcctcatctcccagctgttcaccttctcacctcgccggtacgagacggtgcaagactgcaattgctcactctatcccggccacgtatcaggtcatcgcatggcttgggatatgatgatgaactggtcgcctacaacagccttagtggtatcgcagttactccggatcccacaagccatcgtggacatggtgacaggggcccactggggggtcttggcgggtctcgcctattactccatggtggggaactgggctaaggtcttgattgtgatgctactcttttccggcgttgacggcgccactcgcctgtcagggggggcggcaggtcgtgatacccgcggcttcgcggccctcttccagccagggtcagctcagaacatccagcttataaactccaacggcagctggcacgtcaacaggacagccctgaattgcaatgacaccctccacactgggttcattgccgggctgctctacacaaccaaattcaactcgtccgggtgcccagggcgcctggccagctgccgccccattgacaagttcgcccaggggtggggtcccatcacttatgctgagccaggagcctcggaccagaggccctattgctggcactacgcgcctcggccgtgcggtattgtacccgcgtcgcaggtgtgtggtccagtatattgcttcaccccaagccccgtcgtggtgggtacgaccgatcgctccggtgcccccacgtatacctggggggagaatgagacggacgtgctacttctcaacaacacacggccgccgcaaggcaactggttcggctgtacatggatgaatagcaccgggttcaccaagacgtgtggggcccctccgtgcaacatcggggggagcggcaacaacaccttgatctgccctacggattgcttccggaagcaccccgaggccacttacatcaaatgcggctcggggccgtggttgacacctaggtgtctagttgattacccatacaggctttggcactacccctgcaccgtcaactttaccatcttcaagatcaggatgtatgtggggggcgtggagcacagactcactgccgcatgcaattggactcgaggagagcgttgcgatttggaggacagggatagatcggaacttagcccactgttactctctacaacggagtggcagatactgccctgttccttcaccaccctaccggctttgtccactggtttgattcatctccatcagaacattgtggacgtacaatacctgtacggtgtagggtcagcggttgtctccattgcgatcaaatgggagtacgtcgtgctgctctttctcctcctggcggacgcacgcttctgcgcctgcttgtggatgatgctgctgatagcccaggctgaggccgccttagagaacctggtgatcctcaatgcagcgtccgtggccggagcccgtggcattctctccttccttgtgttcttctgtgctgcctggtacatcaagggcaaactggtccctggggcggcatatgccctctacggtgtatggccgctgcttctgctcctgctggcgttaccaccacgagcatacgcctttgaccgggaaatggctgcatcgtgcggaggcgcggttttcataggtctgatgcttctgaccttgtcaccacactataaggcactcctcgccaggcttatatggtggttacaatattttatcaccagggccgaggcgcacttgcaagtgtggatccccccccttaacgttcgggggggccgcgatgccattatcctcctcacatgtgcggtccattcagagctaatttttgaaatcaccaaaatcttgctcgccatgcttggtccgctcatgatgctccaggctggcctaattagagtgccgtactttgtgcgcgctcaagggcttattcgtgcgtgctt

SEQ ID NO: 44 - protein, 742 aa, Hepatitis C virus
LLALLSCLTVPTSAYEVRNVSGMYQVTNDCSNSSIVYEAADVIMHTPGCVPCVRESNLSRCWVALTPTLAARNSSIPTTTIRRHVDLLVGASAFCSAMYVGDLCGSVFLISQLFTFSPRRYETVQDCNCSLYPGHVSGHRMAWDMMMNWSPTTALVVSQLLRIPQAIVDMVTGAHWGVLAGLAYYSMVGNWAKVLIVMLLFSGVDGATRLSGGAAGRDTRGFAALFQPGSAQNIQLINSNGSWHVNRTALNCNDTLHTGFIAGLLYTTKFNSSGCPGRLASCRPIDKFAQGWGPITYAEPGASDQRPYCWHYAPRPCGIVPASQVCGPVYCFTPSPVVVGTTDRSGAPTYTWGENETDVLLLNNTRPPQGNWFGCTWMNSTGFTKTCGAPPCNIGGSGNNTLICPTDCFRKHPEATYIKCGSGPWLTPRCLVDYPYRLWHYPCTVNFTIFKIRMYVGGVEHRLTAACNWTRGERCDLEDRDRSELSPLLLSTTEWQILPCSFTTLPALSTGLIHLHQNIVDVQYLYGVGSAVVSIAIKWEYVVLLFLLLADARFCACLWMMLLIAQAEAALENLVILNAASVAGARGILSFLVFFCAAWYIKGKLVPGAAYALYGVWPLLLLLLALPPRAYAFDREMAASCGGAVFIGLMLLTLSPHYKALLARLIWWLQYFITRAEAHLQVWIPPLNVRGGRDAIILLTCAVHSELIFEITKILLAMLGPLMMLQAGLIRVPYFVRAQGLI

SEQ ID NO: 45 - DNA, 490 bp, Hepatitis C virus
  CDS (1)..(489)
ctcttggctctgctgtcctgtctgaccatcccagcttccgcttatgaagtgcgcaacgtgtccggaatataccatgtcacgaacgactgctccaactcaagcattgtgtatgaggcagcggacgtgatcatgcatacccccgggtgcgtgccctgtgttcgggagggtaacgcctcccgttgttgggcagcgctcactcccacgctcgcggtcgggaatgccagcgtccccactaaggcaatacggcgccacgtcgatctgcttgttgggacggctgctttctgctccgccatgtacgtgggggatctctgcggatacatcgccaaactcctgctcgccacactcggtctgctcatggtgctccaggctgccatagctagagtgccgtacttcgtacgcactcaggggctcattcgtgtgtgtatgttagtgcggaaagtcgccgggggtcactatgcccagatggccttcatcaagctggccgcactga

SEQ ID NO: 46 - protein, 163 aa, Hepatitis C virus
LLALLSCLTIPASAYEVRNVSGIYHVTNDCSNSSIVYEAADVIMHTPGCVPCVREGNASRCWAALTPTLAVGNASVPTKAIRRHVDLLVGTAAFCSAMYVGDLCGYIAKLLLATLGLLMVLQAAIARVPYFVRTQGLIRVCMLVRKVAGGHYAQMAFIKLAAL

SEQ ID NO: 47 - DNA, 1018 bp, Hepatitis C virus
  CDS (229)..(1017)
ccaggaccccccctcccgggagagccatagtggtctgcggaaccggtgagtacaccggaattgccaggacgaccgggtcctttcttggattaacccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcaccatgagcacgaatcctaaaccccaaagaaaaaccaaccgaaacaccaaccgccgtccacaggacgttaagttcccgggcggtggtcagatcgtcggtggagtttacctgttgccgcgcaggggccccaggttgggtgtgcgcgcgactaggcagacttccgagcggtcgcagcctcgtggaaggcgacaacctatccccaaggttcgccggcccgagggcagaacctgggctcagcccgggtatccttggcccctctatggcaatgagggcttggggtgggcaggatggctcctgtcaccccgtggctcccggcctagttggggccccacggacccccggcgtaggtcgcgtaatttgggtaaggtcatcgataccctcacatgcggcttcgccgacctcatggggtatattccgcttgtcggcgcccctttaggaggcgctgccagggccctggcacatggtgtccgggttctggaggacggcgtgaattctgcaacagggaatttgcctggttgctctttctctatcttcctcttggctctgctgtcctgtttgaccatcccagcttccgcttatgaagtgcgcacgtgcgcggtccatccagagccaatctttgacatcaccaacctcctgctcgccatactcggcccgctcatggtgctccaggctggcataactagagtgccgtacttcgtacgcgctcaagggctcattcgtgcatgcatgttagtgcggaaaacgcctgggggtcattatgtccaaatggccctcatgaggctggccgcactga

SEQ ID NO: 48 - protein, 263 aa, Hepatitis C virus
MSTNPKPQRKTNRNTNRRPQDVKFPGGGQIVGGVYLLPRRGPRLGVRATRQTSERSQPRGRRQPIPKVRRPEGRTWAQPGYPWPLYGNEGLGWAGWLLSPRGSRPSWGPTDPRRRSRNLGKVIDTLTCGFADLMGYIPLVGAPLGGAARALAHGVRVLEDGVNSATGNLPGCSFSIFLLALLSCLTIPASAYEVRTCAVHPEPIFDITNLLLAILGPLMVLQAGITRVPYFVRAQGLIRACMLVRKTPGGHYVQMALMRLAAL

SEQ ID NO: 49 - DNA, 32 bp, Artificial Sequence
  Description of Artificial Sequence Synthetic primer
cgcggatccttagtcctccagaacccggacac

SEQ ID NO: 50 - DNA, 19 bp, Artificial Sequence
  Description of Artificial Sequence Synthetic primer
tgcacggtctacgagacct

SEQ ID NO: 51 - DNA, 20 bp, Artificial Sequence
  Description of Artificial Sequence Synthetic primer
tagtggtctgcggaaccggt

SEQ ID NO: 52 - DNA, 25 bp, Artificial Sequence
  Description of Artificial Sequence Synthetic primer
gccgcatgtaagggtatcgatgacc

SEQ ID NO: 53 - DNA, 25 bp, Artificial Sequence
  Description of Artificial Sequence Synthetic primer
ggtcatcgatacccttacatgcggc

SEQ ID NO: 54 - DNA, 32 bp, Artificial Sequence
  Description of Artificial Sequence Synthetic primer
gcgaattcttatcagaagaactcgtcaagaag

SEQ ID NO: 55 - DNA, 26 bp, Artificial Sequence
  Description of Artificial Sequence Synthetic primer
gcctattggcctggagtgtttagctc

SEQ ID NO: 56 - DNA, 29 bp, Artificial Sequence
  Description of Artificial Sequence Synthetic primer
atggcgttagtatgagtgtcgtgcagcct

SEQ ID NO: 57 - DNA, 25 bp, Artificial Sequence
  Description of Artificial Sequence Synthetic primer
agccgcatgtaagggtatcgatgac

SEQ ID NO: 58 - DNA, 23 bp, Artificial Sequence
  Description of Artificial Sequence Synthetic primer
tggttcggctgyacatggatgaa

SEQ ID NO: 59 - DNA, 26 bp, Artificial Sequence
  Description of Artificial Sequence Synthetic primer
ggrtagtgccaragcctgtatgggta

SEQ ID NO: 60 - DNA, 31 bp, Artificial Sequence
  Description of Artificial Sequence Synthetic primer
tcgggcacgagacaggctgtgatatatgtct

SEQ ID NO: 61 - DNA, 30 bp, Artificial Sequence
  Description of Artificial Sequence Synthetic primer
atcgtcttcacgcagaaagcgtctagccat

SEQ ID NO: 62 - DNA, 31 bp, Artificial Sequence
  Description of Artificial Sequence Synthetic primer
gccagccccctgatgggggcgacactccacc

SEQ ID NO: 63 - DNA, 30 bp, Artificial Sequence
  Description of Artificial Sequence Synthetic primer
aatcatatgtctttgaggtttaggatttgt

SEQ ID NO: 64 - DNA, 29 bp, Artificial Sequence
  Description of Artificial Sequence Synthetic primer
gacatatgattgaacaagatggattgcac

SEQ ID NO: 65 - DNA, 33 bp, Artificial Sequence
  Description of Artificial Sequence Synthetic primer
gtcctgcaggccagccccctgatgggggcgaca

SEQ ID NO: 66 - DNA, 34 bp, Artificial Sequence
  Description of Artificial Sequence Synthetic primer
gacctgcaggttatcagaagaactcgtcaagaag

SEQ ID NO: 67 - DNA, 50 bp, Artificial Sequence
  Description of Artificial Sequence Synthetic primer
gccttaattaatacgactcactataggccagccccctgatgggggcgaca

SEQ ID NO: 68 - DNA, 76 bp, Artificial Sequence
  Description of Artificial Sequence Synthetic primer
tctagtcgacggccagtgaattgtaatacgactcactatagggcggccagccccctgatgggggcgacactccacc

SEQ ID NO: 69 - DNA, 28 bp, Artificial Sequence
  Description of Artificial Sequence Synthetic primer
aggcctgtgaagacgctctcccagaact

SEQ ID NO: 70 - DNA, 21 bp, Artificial Sequence
  Description of Artificial Sequence Synthetic primer
ggtgatgaccttggtctccat

SEQ ID NO: 71 - DNA, 32 bp, Artificial Sequence
  Description of Artificial Sequence Synthetic primer
gcttagaggctagtgatgatgcaaccaagtac

SEQ ID NO: 72 - DNA, 24 bp, Artificial Sequence
  Description of Artificial Sequence Synthetic primer
ggcgaccgcatagtagtttccata

SEQ ID NO: 73 - DNA, 32 bp, Artificial Sequence
  Description of Artificial Sequence Synthetic primer
ctggaggacggcgtgaactatgcaacagggaa

SEQ ID NO: 74 - DNA, 32 bp, Artificial Sequence
  Description of Artificial Sequence Synthetic primer
ggaacttgcccggttgctctttctctatcttc

SEQ ID NO: 75 - DNA, 21 bp, Artificial Sequence
  Description of Artificial Sequence Synthetic primer
tggggcaagatggttataaac

SEQ ID NO: 76 - DNA, 31 bp, Artificial Sequence
  Description of Artificial Sequence Synthetic primer
ggggtaagatggttataaacgtacgtacctg

SEQ ID NO: 77 - DNA, 31 bp, Artificial Sequence
  Description of Artificial Sequence Synthetic primer
ataatgacccccggcgactttccgcactaac

SEQ ID NO: 78 - DNA, 24 bp, Artificial Sequence
  Description of Artificial Sequence Synthetic primer
tgacatcagcatgtctcgtgacca

SEQ ID NO: 79 - DNA, 26 bp, Artificial Sequence
  Description of Artificial Sequence Synthetic primer
cttgaaaaagccctggattgtcagat

SEQ ID NO: 80 - DNA, 20 bp, Artificial Sequence
  Description of Artificial Sequence Synthetic primer
acatgatctgcagagaggcc

SEQ ID NO: 81 - DNA, 26 bp, Artificial Sequence
  Description of Artificial Sequence Synthetic primer
ctacggggcctgttactccattgaac

SEQ ID NO: 82 - DNA, 35 bp, Artificial Sequence
  Description of Artificial Sequence Synthetic primer
acatgatctgcagagaggccagtatcagcactctc

SEQ ID NO: 83 - DNA, 73 bp, Artificial Sequence
  Description of Artificial Sequence Synthetic primer
tctagtcgacggccagtgaattgtaatacgactcactctagggcggcggggtcgggcwcgngacabgctgtga

SEQ ID NO: 84 - DNA, 31 bp, Artificial Sequence
  Description of Artificial Sequence Synthetic primer
tctccattgggctgaacaccacaggctccac

SEQ ID NO: 85 - DNA, 31 bp, Artificial Sequence
  Description of Artificial Sequence Synthetic primer
ggggagaggtggtcatagatgtaagtgccgg

SEQ ID NO: 86 - DNA, 30 bp, Artificial Sequence
  Description of Artificial Sequence Synthetic primer
catagatgtaagtgccggtccacctgccta

SEQ ID NO: 87 - DNA, 30 bp, Artificial Sequence
  Description of Artificial Sequence Synthetic primer
ctcctgcgaggtgtctcaccagggtacaca

SEQ ID NO: 88 - DNA, 29 bp, Artificial Sequence
  Description of Artificial Sequence Synthetic primer
agcagagcgtgagctctgacgaagtatgg

SEQ ID NO: 89 - DNA, 32 bp, Artificial Sequence
  Description of Artificial Sequence Synthetic primer
ggaatctacccggttgctctttttctatcttc

SEQ ID NO: 90 - DNA, 32 bp, Artificial Sequence
  Description of Artificial Sequence Synthetic primer
ctggaagacgggataaattatgcaacagggaa

SEQ ID NO: 91 - DNA, 24 bp, Artificial Sequence
  Description of Artificial Sequence Synthetic primer
ctcgcaagcaccctatcagccagt

SEQ ID NO: 92 - DNA, 20 bp, Artificial Sequence
  Description of Artificial Sequence Synthetic primer
aggcattgagcgggtttatc

SEQ ID NO: 93 - DNA, 38 bp, Artificial Sequence
  Description of Artificial Sequence Synthetic primer
ctagactcgagtcgacatcgtttttttttttttttttt

SEQ ID NO: 94 - DNA, 20 bp, Artificial Sequence
  Description of Artificial Sequence Synthetic primer
atcttagccctagtcacggc

SEQ ID NO: 95 - DNA, 20 bp, Artificial Sequence
  Description of Artificial Sequence Synthetic primer
ctagactcgagtcgacatcg

SEQ ID NO: 96 - DNA, 30 bp, Artificial Sequence
  Description of Artificial Sequence Synthetic primer
ctagctgtgaaaggtccgtgagccgcatga

SEQ ID NO: 97 - DNA, 42 bp, Hepatitis C virus
  CDS (1)..(42)
tgctcaaacaacagcatcacctggcagctcactgacgcagtt

SEQ ID NO: 98 - protein, 14 aa, Hepatitis C virus
CSNNSITWQLTDAV

SEQ ID NO: 99 - DNA, 69 bp, Hepatitis C virus
  CDS (1)..(69)
gtcattctacacccacgccttgtgtttgaggtcacgaaatggtgctcgaacaacagcatcacctggcag

SEQ ID NO: 100 - protein, 23 aa, Hepatitis C virus
VILHPRLVFEVTKWCSNNSITWQ

SEQ ID NO: 101 - DNA, 27 bp, Hepatitis C virus
  CDS (1)..(27)
cgccttgtgtttgaagtcacaaaatgg

SEQ ID NO: 102 - protein, 9 aa, Hepatitis C virus
RLVFEVTKW

SEQ ID NO: 103 - DNA, 42 bp, Hepatitis C virus
  CDS (1)..(42)
atggggaacacatcacggtgctggataccagtctcaccaaag

SEQ ID NO: 104 - protein, 14 aa, Hepatitis C virus
MGNTSRCWIPVSPK

SEQ ID NO: 105 - DNA, 69 bp, Hepatitis C virus
  CDS (1)..(69)
gagtgggcaccatccatgcaggcgcgcggcggccgtgatggcgtgggaaatacatcacgatgctggata

SEQ ID NO: 106 - protein, 23 aa, Hepatitis C virus
EWAPSMQARGGRDGVGNTSRCWI

SEQ ID NO: 107 - DNA, 27 bp, Hepatitis C virus
  CDS (1)..(27)
atgcaggcgcgcggtggccgcgatggc

SEQ ID NO: 108 - protein, 9 aa, Hepatitis C virus
MQARGGRDG

SEQ ID NO: 109 - DNA, 42 bp, Hepatitis C virus
  CDS (1)..(42)
gttctccatcttcctggatgcgtcccatgtgagaatgataat

SEQ ID NO: 110 - protein, 14 aa, Hepatitis C virus
VLHLPGCVPCENDN

SEQ ID NO: 111 - DNA, 69 bp, Hepatitis C virus
  CDS (1)..(69)
  modified_base (69)..(69): a, c, g, or t
gtccgaggggggcgtgacgggatcatctgggtggctgtcattgttctccaccttcctggatgtgttccn

SEQ ID NO: 112 - protein, 23 aa, Hepatitis C virus
VRGGRDGIIWVAVIVLHLPGCVP

SEQ ID NO: 113 - DNA, 24 bp, Hepatitis C virus
  CDS (1)..(24)
gggatcatctgggcggctggcatt

SEQ ID NO: 114 - protein, 8 aa, Hepatitis C virus
GIIWAAGI

(19) United States
(12) Patent Application Publication
(10) Pub. No.: US 2009/0170063 A1
(43) Pub. Date: Jul. 2, 2009

(54) Hcv rna having novel sequence

(75) Inventors: Shintaro Yagi, Saitama (JP); Kenjiro Yamaguchi, Saitama (JP); Kenichi Mori, Saitama (JP); Eiji Tanaka, Nagano (JP); Kendo Kiyosawa, Nagano (JP); Noboru Maki, Saitama (JP)

Correspondence Address: FOLEY AND LARDNER LLP;SUITE 500, 3000 K STREET NW, WASHINGTON, DC, 20007, US

(73) Assignee: Advanced Life Science Institute, Inc.

(21) Appl. No.: 11/630,374

(22) PCT Filed: Jun. 24, 2005
(86) PCT No.: PCT/JP05/12162

§ 371 Date: Dec. 22, 2006

(30) Foreign Application Priority Data
Jun. 25, 2004 (JP) 2004-188543
Jun. 28, 2004 (JP) 2004190144
Sep. 24, 2004 (JP) 2004-277677

(51) Int. Cl. C12Q 1/70; C07H 21/00; C12N 5/10; C12N 7/00; C07K 14/005; C07K 16/08

(52) U.S. Cl. 435/5; 536/23.72; 435/325; 435/235.1; 530/350; 530/387.1

(57) ABSTRACT

A truncated form hepatitis C virus gene wherein part of the gene region encoding from the core protein to the NS2 protein of hepatitis C virus has been deleted while retaining the translation frame. In particular, the gene according to claim 1 wherein said part of the gene region is present in a region encoding at least the E1 protein and the E2 protein.

### TECHNICAL FIELD

The present invention relates to the genome of type C chronic hepatitis virus (hereinafter referred to as “HCV”).

### BACKGROUND ART

HCV is a causative agent of chronic hepatitis C, and it is estimated according to WHO statistics that 170 million people are infected throughout the world. Though, unlike other viral hepatitis, HCV causes only a relatively mild symptom at the early stage of infection, the infection progresses to a chronic type at a high frequency, and after a certain asymptomatic period, develops chronic hepatitis. As the infection is prolonged, the condition becomes aggravated to cirrhosis, which leads to hepatic carcinoma at a high frequency. Hepatitis viruses are responsible for 95% of hepatic cancer, and most (80%) of it is believed to be caused by HCV.

HCV is transmitted through blood, blood components and, less frequently, body fluid components. Since testing methods for HCV have been introduced into screening at the time of blood transfusion, novel cases of posttransfusion type C hepatitis are very rare now in advanced countries. Furthermore, due to progress in medical technology, transmission due to malpractices is rare, and in Japan the appearance of new patients has been almost suppressed. Based on the epidemiological survey, however, the number of HCV carriers is estimated to be not less than 1.7 million in Japan, and most of them are 40 years of age or older and the infection tends to be prolonged. Thus, there is a grave concern for increases in the number of cases of hepatic carcinoma in the future.

The risk of hepatic carcinoma is highly associated with the state of fibrosing of the liver, and the more progressed the fibrosing is, the higher the incidence of hepatic carcinoma becomes. The state of fibrosing of the liver is usually diagnosed by combining the determination of blood concentrations of Type IV collagen and hyaluronic acid, platelet counts in the blood, diagnostic imaging (abdominal echo check) etc., and, for definite diagnosis, a liver biopsy is carried out. However, liver biopsy poses a major problem since it poses a great burden on the patients, and more patient-oriented diagnostic methods are being required.

HCV, comprising an about 9,600-base plus strand RNA genome, is a virus classified into the family Flaviviridae, the genus Flavivirus, and is thought to be transmitted through the blood and blood components and to propagate in the liver. The analysis of the gene sequence suggests the presence of at least six genotypes. After infection, the about 9,600-base genome functions as mRNA in the host cell, and a polyprotein of a stretch of about 3,000 amino acid length is synthesized, which is cleaved with a signal peptidase, signal peptidyl peptidase in the host cells, and a protease encoded by the HCV genome. As a result, 10 types of proteins such as core, E1, E2, p7, NS2, NS3, NS4A, NS4B, NS5A and NS5B are produced. In addition to this translation frame (open reading frame), there are untranslated regions (UTR) at the 5′-end and the 3′-end and these regions are responsible for the functions of translation control and genome replication control.

Among them, core, E1 and E2 are structural proteins that constitute the virus, and it is believed that the virus genome is packaged by the core protein to form a capsid, and surrounded by the E1 and E2 proteins anchored to the lipid bilayer membrane to form a virus particle (virion). The function of p7 is unknown, but is reported to be essential for the viral propagation. NS2 is a metal protease and is necessary for the cleavage of itself, but its other functions are unknown. It is believed that a complex comprising of the protein NS3 to NS5B forms a RNA replication unit together with host proteins to perform the replication of genomic RNA.

For the diagnosis of HCV infection, methods of detecting antibodies against proteins produced by HCV contained in the biological components (blood, serum, plasma etc.) of patients are widely used. However, the mere presence of antibody cannot identify whether HCV is active or not, and therefore the measurement of amount of virus in the blood is important in diagnosis. For the detection or measurement of virus in the blood, methods of detecting or determining the virus genome, or determining or detecting the core protein produced by HCV are used.

For the treatment of chronic hepatitis C, interferon has been widely used. Due to the recent improvement of the drugs and the administration regimen such as the combined therapy with ribavirin, the ratio of complete remission resulted from HCV eliminated from the body are increasing. However, the ratio of complete remission is still about 50%. Also interferon administration may cause severe side effects, and thus may not be used for elderly patients. Therefore, the development of more effective therapeutic methods or therapeutic agents is being required.

With regard to the relation of the interferon therapy and HCV, the tendency that the more the amount of the virus in the blood, the higher the resistance to the interferon therapy is, is recognized. There are also cases in which the difference of sensitivity to interferon according to genotypes is observed, and the genotype 2 in particular shows a relatively high sensitivity to interferon treatment. However, there are no definite relationship between the amount of virus in the blood and the degree of aggravation of hepatitis, or between the genotype and the grave condition of hepatitis. Thus, there are no virus markers that represent the grave condition of hepatic diseases.

HCV infects humans through the blood or blood components. In organisms other than humans, it infects anthropoids (chimpanzees), and the infection leads to the onset of hepatitis and sometimes to chronic hepatitis. In other experimental animals that are easy to keep, none are known to be infected with HCV at a high frequency.

On the other hand, it is reported that HCV could be infected to human- and monkey-derived cells in vitro and could be propagated. However, both of the infection ratio and the propagation efficiency were low. Thus, it is very difficult to infect and propagate HCV in vitro.

It was found in recent years that by transfecting the RNA containing a portion of synthesized HCV genome (subgenomic RNA) in vitro and a drug resistant marker into an established human hepatoma-derived cell, a cell in which the RNA is autonomously replicating in the cell can be isolated though at a low frequency. Since this has been reproduced in many laboratories and the cells can be maintained relatively easily, it came to be widely used in research. In these replicons the structural protein part of HCV has been artificially deleted, suggesting that the replication unit composed of proteins produced by the non-structural region has information necessary and adequate for the replication of the plus strand RNA of HCV in the cell.

The propagation of HCV in the liver has been indicated by the detection of proteins produced by HCV in the liver and the detection of HCV RNA in the liver. Also, by eliminating HCV transiently or permanently by the interferon therapy, the subsequent condition of hepatitis is alleviated, which clearly indicates that HCV is related with the development of pathology in the liver. However, much has yet to be elucidated on the mode of presence of HCV in the liver.

Thus, the mechanism of development and progress of pathology in which HCV infection leads to the onset of hepatitis, which turns into a prolonged chronic type and the disease condition aggravates, finally leading to hepatic carcinoma has not been elucidated. The extensive research proposes hypotheses on the mechanism of development and aggravation of pathological conditions by allowing each HCV protein to be recombinantly expressed in cultured cells and analyze the state of the expressing cells. Since there are no appropriate models of HCV infection and propagation, the hypotheses cannot be confirmed.

Patent document 1: Japanese Unexamined Patent Publication (Kokai) No. 2001-17187

Non-patent document 1: Science, Vol. 277, pp. 570-, 1997

Non-patent document 2: Journal of Virology, Vol. 76, pp. 4008-4021, 2002

Non-patent document 3: Science, Vol. 285, pp. 110-, 1999

Non-patent document 4: Science, Vol. 290, pp. 1972-, 2000

Non-patent document 5: Hepatology, Vol. 29, pp. 223-229, 1999

Non-patent document 6: Journal of Virology, Vol. 77, pp. 2134-2146, 2003

Non-patent document 7: Res. Virol., Vol. 144, pp. 275-279, 1993

Non-patent document 8: Journal of Virology, Vol. 75, pp. 4614-4624, 2001

Non-patent document 9: PNAS, Vol. 29, 14416-14421, 2002

Non-patent document 10: Current Opinion in Infectious Disease, Vol. 14, pp. 743-747

Non-patent document 11; Journal of Viral Hepatitis, Vol. 6, pp. 35-47, 1999

Non-patent document 12: Clinical Chemistry, Vol. 43, pp. 1507-1522, 1997

Non-patent document 13: Journal of General Virology, Vol. 81, pp. 1631-1648, 2000

### DISCLOSURE OF THE INVENTION

Though HCV is thought to replicate itself and propagate in the liver, its mode of presence in the liver has not been analyzed. Accordingly there is very little information on how HCV is replicated in the liver and what molecules are appropriate as the target of therapeutic agents for HCV. Consequently, the therapy of HCV is carried out on a trial and error basis. Therefore, in order to develop the therapeutic agents efficiently, it is necessary to identify HCV genomic RNA that is being actively replicated in the liver of the patient. Thus, the present invention relates to HCV genomic RNA that is being replicated and propagating in the liver, and intends to provide HCV genomic RNA that serves as an appropriate target for the therapeutic agents.

Though the propagation of HCV aggravates the condition of hepatitis, there are no virus markers that indicate the condition of hepatitis. Virus markers that indicate the condition of hepatitis are being required. The present invention intends to provide a virus marker that indicates the symptom and condition of hepatitis.

Since there are no experimental animals that are easy to handle and that are able to be infected with HCV or no in vitro culture systems in which is infected with HCV at a high frequency and replicates HCV efficiently, it is difficult to carry out efficient drug screening. These represent the reasons that make the development of HCV-specific therapeutic agents difficult. For example, for the widely used interferon therapy, methods of treatment have been directly developed and improved with patients as the subject, and have posed a great burden on the patients. Thus, more patient-oriented methods for developing pharmaceutical drugs are needed. In order to resolve this problem, models of HCV infection and propagation that can be widely used are required in the development of effective therapeutic agents and therapeutic methods. One of the objectives of the present invention is to provide a model of HCV infection and propagation that can be used in the development and screening etc. of therapeutic agents and therapeutic methods.

As such a propagation model, a system that employs a subgenomic replicon has been used. Thus, by transfecting RNA having an appropriate structure encoding a HCV subgenome and an appropriate drug resistant marker into a human hepatoma-derived cell and selecting the cell retaining the RNA by drug resistance, a HCV subgenomic RNA replicon that autonomously propagates in the cell can be obtained. Using this replicon and the cell retaining the replicon, the screening of drugs for HCV is under way. However, this method has serious problems.

One of them is that the genome of subgenomic RNA replicon is composed of a structure different from the original HCV genomic RNA. Thus, the subgenomic RNA has only information derived from the nonstructural protein of the HCV genome. In order to improve this, a replicon comprising all the translated region of the HCV protein and a drug resistant marker was established. In cells that retain this replicon, all proteins encoded by the HCV genome should be produced, and thus it was expected that HCV particles should be released outside the cell. However, no HCV particles were released outside the cell. Thus, even with such an improvement, it appears to be incomplete as a propagation system of HCV.

Also, the above subgenomic replicon comprising the nonstructural region has the internal ribosome entry site (IRES) of the 5′-untranslated region and the neomycin resistant gene downstream of the gene of portion of the core, and, downstream thereto, the internal ribosome entry site (IRES) of the encephalomyocarditis virus (EMCV), a region encoding of nonstructural protein of HCV and the 3′-untranslated region. Also, the improved replicon having all of the translated region is one in which the gene region encoding the nonstructural protein of the subgenomic replicon has been replaced with a region encoding all the translated region of HCV, and the fundamental structure of these replicons is essentially the same.

Unlike the structure of the original HCV, these replicons have two IRES's (HCV IRES and EMCV IRES), and are different from the structure of HCV genome in the living body. This difference in structure is possibly one of reasons that HCV particles can not be released outside the cell. Furthermore, replicons having such a structure may be replicated in a mechanism different from the virus genome replication in the living body.

From the foregoing, it is desirable that the replicon system of HCV is a replicon system that employs RNA having the original HCV genome structure in the living body. The present invention intends to provide a replicon comprising a sequence and a structure that is actually replicated in the liver.

A further serious problem is the so-called adaptive mutation, i.e. the presence of mutation which is peculiar to the experimental system of the subgenomic replicon. When a HCV replicon which is functional in vitro was collected and analyzed, a plurality of mutations were found that were not originally present in NS3 or NS5A. This mutation is important for the effective replication and influences the efficiency of replication. However, it is known that mutation suitable for replication is undesirable for the propagation of HCV. By inoculating an in vitro-synthesized HCV genome directly into the liver of a chimpanzee, the chimpanzee is infected with HCV.

When a mutation which is important for the subgenomic replicon is introduced into the HCV RNA synthesized in vitro, the infectivity to chimpanzees retained by the original sequence disappeared. Furthermore, there are no sequences that have infectivity to chimpanzees and that are replicated and propagated in vitro in the cell. Therefore, sequences that came to be replicated in vitro have lost the function of replicating and propagating in vivo, and thus do not retain the ability of replicating RNA and propagating owned by the original HCV. They must be sequences capable of replicating and propagating both in vivo and in vitro. Thus, the replicon provided by the present invention having the HCV genomic RNA sequence can replicate both in vivo and in vitro.

The present inventors have isolated cDNA to the HCV genomic RNA in the liver of a patient and have determined the entire structure. By analyzing the sequence determined, the isolated cDNA was found to have a sequence entirely different from the structure of the HCV genomic RNA so far reported.

The genomic RNA having a new structure is characterized in that part or all of the structural protein regions of the HCV genomic RNA previously reported are deleted, and it was found that the flanking sequences of the deleted portion are connected while retaining the translation frame (reading frame) of the original HCV genome, and that this HCV genome encodes one stretch of a novel polypeptide. The one stretch of polypeptide can generates part of the structural protein and all of the nonstructural proteins of the original HCV.

Since the HCV polyprotein encoded by this genome retains a cleavage site by the intracellular signal peptidase and a cleavage sequence by its own protease, it is estimated to undergo processing. In fact, when the novel HCV genomic cDNA set forth in SEQ ID NO: 1 was expressed in a mammalian cell and the product was analyzed, the core protein was normally processed, the E1 and NS2 proteins were expressed as a fusion protein and processed with NS3, and NS3 was processed to a molecule of the original size.

This HCV genomic RNA is called a truncated form (TF). In contrast, the previously reported HCV RNA containing all of the structural genes is called a full-length form (FLF). Biopsy samples and serums from a plurality of patients with chronic hepatitis C were analyzed. From a plurality of patients, a TF HCV genome having the common characteristics was detected. The characteristic is that part or all of sequence of the structural protein has been deleted, but the latter half of the NS2 sequence and after are retained, and that the remaining sequence may be expressed in such a form as to retain the translation frame that is inferred from FLF.

Thus, the present invention provides:

(1) A truncated form hepatitis C virus gene wherein part of the structural protein-coding region has been retained and, while retaining one translation frame, part of a region encoding from the core protein to the NS2 protein has been deleted in the hepatitis C virus gene;

(2) The truncated form hepatitis C virus gene according to (1) wherein part of the structural protein-coding region has been retained and, while retaining one translation frame, at least a region encoding the amino acid sequence at the junction the E1 protein and the E2 protein has been deleted in the hepatitis C virus gene;

(3) A truncated form hepatitis C virus gene wherein part of the E1 protein-coding region, the E2 protein-coding region, the P7 protein-coding region and part of the NS2 protein-coding region have been deleted while retaining the translation frame in the hepatitis C virus gene;

(4) The gene according to any of the above (1) to (3), said gene having 5′UTR and 3′UTR; and

(5) The gene according to any one of the above (1) to (3), said gene having 5′UTR, a protein-coding region downstream from NS3, and 3′UTR.

Though hepatitis C virus genome is RNA, the hepatitis C virus gene of the present invention may be used whether it is RNA or DNA.

In other words, the present invention provides (a) a truncated form hepatitis C virus gene wherein part of the E1 protein-coding region, the E2 protein-coding region, the P7 protein-coding region and part of the NS2 protein-coding region have been deleted while retaining the translation frame in the hepatitis C virus gene.

The present invention also provides the truncated form hepatitis C virus gene according to the above (a), said gene having all or part of a region encoding from the 5′-untranslated region to the core protein which is a structural protein and all or part of a region encoding from a region encoding two transmembrane domains at the latter part of NS2 which is a nonstructural protein to the 3′-untranslated region.

Furthermore, the present invention provides the truncated form hepatitis C virus gene according to the above (a), said gene having all or part of No. 1 to No. 914 and all or part of No. 3001 and after of the nucleic acid sequence of the hepatitis C virus gene.

On the other hand, the analysis of the subgenomic replicon demonstrated that information necessary for RNA replication is encoded in the region of the nonstructural protein at NS3 and after. Thus, the TF genome has all the information of the nonstructural protein required for RNA replication. Furthermore, since the TF genome is preferentially detected in some liver biopsy samples, it can be seen that the TF genome is autonomously replicating in the liver. In addition, when TF-containing RNA is transfected in vitro into the cell, it is replicated in the cell. Thus, the TF HCV genomic RNA functions as a replicon. These results revealed that the HCV genomic RNA provided by the present invention can be replicated both in the liver and in vitro.

Thus, the present invention also provides:

(6) A replicon gene of a truncated form hepatitis C virus gene wherein part of region of the gene encoding from the core protein to the NS2 protein of the hepatitis C virus that autonomously replicates in the cell has been deleted while retaining the translation frame, or a truncated form hepatitis C virus gene wherein the deleted region is present at a region encoding at least the E1 protein and the E2 protein.

The present invention also provides:

(7) A replicon gene in which a selection marker gene has been connected to the replicon gene according to the above (3).

Alternatively, it provides:

(8) a cell in which the above replicon gene is replicated.

TF propagates more advantageously than FLF in the liver, and the patients exhibit severe symptoms of hepatitis. The appearance of TF is deeply associated with symptoms of hepatitis, and thus the suppression, inhibition and elimination of the propagation of TF provide an effective therapeutic method. Thus, drugs that effectively suppress or alleviate the symptoms of chronic hepatitis C are preferably developed with TF as the target. Thus, it is obvious that the screening of pharmaceuticals with a replicon using TF is suitable for the development of pharmaceuticals for suppressing, alleviating or eliminating the progress of hepatitis to severe forms.

Thus, the present invention further provides:

(9) A method of screening drugs using a cell in which a truncated form gene is replicated, a method of evaluating the efficacy of drugs, and a method of producing drugs by evaluating the efficacy;

(10) A cell in which a vector having integrated a truncated form gene therein is retained and in which the protein is being expressed; and

(11) A method of diagnosing HCV using a cell in which a replicon containing a truncated form gene is being replicated or a protein produced by the cell.

The TF genome can also be detected in the blood. Thus, this HCV genomic RNA is replicated in the liver and exits as VLP in the blood. Therefore, for the detection of the TF genome, blood or blood components can be used as the sample.

In patients in whom TF is detected, there can be observed cases in which TF is the dominant form among the liver RNAs. This indicates that TF is more suitable for RNA replication in the liver than FLF. Analysis of liver biopsy and serum from many patients revealed that patients in which TF is the dominant form develop severe hepatitis in many cases, and prognosis of the interferon therapy is bad in many of them. This indicates that when TF is the dominant form in the liver, hepatitis is at a severe stage and the effect of the interferon therapy is limited. Thus, the detection and determination of TF provides an index for the state of hepatitis. This means that a method of detecting and determining TF can be applied as a method for diagnosing the state of hepatitis and can be used as a novel virus marker.

Thus, the present invention also provides:

(12) A method of detecting a truncated form gene in the sample. This method also includes methods of detecting the truncated form gene of hepatitis C virus using all methods for detecting the deletion of genes.

As another example, the present invention provides a method of detecting or determining the truncated form gene by amplifying the truncated form gene by PCR with primers designed from sequences from nucleic acids No. 1 to No. 914 and No. 3001 and after of the hepatitis C virus gene.

(13) It also provides a method of determining the quantitative ratio of the truncated form hepatitis C virus gene and the full-length form hepatitis C virus gene in a sample in which they are mixed.

(14) It also provides a method of determining the quantitative ratio by quantitating a gene at the common region of the truncated form gene and the full-length form gene, and quantitating a gene at the region where is deleted in the truncated form gene.

The TF genome in the blood is thought to exist as hepatitis C virus particles or hepatitis C virus-like particles. This is because, unless a certain structure is formed, the TF genome which is RNA is rapidly digested by RNase in the blood and cannot be detected.

Thus the present invention provides:

(15) Hepatitis C virus particles or hepatitis C virus-like particles retaining the truncated form gene.

In the TF genome, a region encoding the structural protein is deleted. Therefore, a novel HCV polyprotein in which a peptide that was encoded in the deleted region is deleted can be produced. As described above, when the HCV genomic cDNA set forth in SEQ ID NO: 1 was expressed in a mammalian cell, a fusion protein of E1 and NS2 was produced.

Thus, the present invention also provides:

(16) A polyprotein of the hepatitis C virus produced from the truncated form gene and a protein processed from the polyprotein. It also provides:

(17) An antibody that specifically recognizes this protein.

By using a novel HCV genomic RNA (TF), a RNA replicon replicating in vivo can be replicated in vitro. By using this replicon, a model of infected cells which are preferentially replicated in the patient's liver and which aggravate hepatitis into a severe form can be constructed. By using this model, the development and screening of pharmaceuticals that suppress and/or inhibit the progress of hepatitis into a severe form can be carried out.

The detection or quantitation of TF is effective for grasping the state of hepatitis, and is useful as a virus marker that indicates the state of hepatitis. Also by a diagnostic method using this marker, the state of hepatitis in patients, monitoring of the effect of drugs, and resistance to drugs can be diagnosed.

### BRIEF EXPLANATION OF THE DRAWINGS

FIG. 1 shows a representative image of electrophoresis of a TF genome detected by RT-PCR from the HCV genomic RNA of the liver tissue. In primer set 1, the cDNA synthetic primer is 3481R, the 1st PCR primer is HClongA1 and 3481R, and the 2nd PCR primer is 85F and 3279R; in primer set 2, the cDNA synthetic primer is 3945R, the 1st PCR primer is 831S and 3945R, and the 2nd PCR primer is 841S and 3759R; in primer set 3, the cDNA synthetic primer is 3945R, the 1st PCR primer is 813S and 3174AS, and the 2nd PCR primer is 841S and 3111AS. The arrow indicates the PCR product of the TF genome.

FIG. 2 shows difference in the structure of the TF genome in a sample in which the TF genome was only detected and D89815 genome. In a nucleic acid sequence encoding each protein of D89815, the core is present at 341-914, E1 at 915-1454, E2 at 1455-2579, P7 at 2580-2778, and NS2 at 2779-3419. For the TF genome obtained from each sample, 5′ UTR is represented by a solid line and the protein-coding region is represented by a gray bar. The deleted region is represented by connecting the bars by a line. The number under the bar of each sample represents the position of a nucleic acid corresponding to D89815.

FIG. 3 shows difference in the structure of the TF genome in a sample in which both the TF genome and the FLF genome were detected and D89815 genome. As in FIG. 2, for the TF genome obtained from each sample, 5′UTR is represented by a solid line and the protein-coding region is represented by a gray bar. The deleted region is represented by connecting the bars by a line. The number under the bar of each sample represents the position of a nucleic acid sequence corresponding to D89815.

FIG. 4 is a graph showing time course in the amount of core antigen in Example 6.

FIG. 5 shows flanking sequences of a gene having the deletion obtained in Example 7.

### BEST MODE FOR CARRYING OUT THE INVENTION

The best mode of the present invention is described below, but it should be noted that the present invention is not limited to it in any way.

The present invention relates to the gene of a novel structure of hepatitis C virus. The normal FLF gene of hepatitis C virus comprises 5′UTR followed by regions encoding the structural proteins of the virus such as the core protein, the E1 protein, the E2 protein and the P7 protein, and the nonstructural proteins such as the NS2 protein, the NS3 protein, the NS4A protein, the NS4B protein, the NS5A protein and the NS5B protein, and 3′UTR.

The hepatitis C virus gene of the present invention is a TF genome in which genes encoding all or part of the structural proteins such as the core protein, the E1 protein, the E2 protein and the P7 protein, and/or part of the NS2 protein, a nonstructural protein, have been deleted. In this TF genome, there is in-frame deletion while retaining the translation frame of the FLF genome, and in the region other than the deleted regions, the normal HCV polyprotein can be produced. Thus, the present invention relates to a hepatitis C virus gene in which part of the region of the gene encoding the polyprotein of hepatitis C virus has been deleted in-frame, and is characterized by the structure of the TF genome.

The TF genome of HCV retains 5′UTR, and it is believed that in many cases at least the gene at No. 3001 and after of the FLF genome of HCV has been retained. In the gene at No. 3001 and after, two transmembrane domains at the C terminal of the NS2 region are present, and proteins of HCV at NS3 and after can be normally produced in the cell from this TF genome (a number indicating the position of the nucleic acid sequence describes the number of the position corresponding to the HCV sequence in GeneBank accession No. D89815 which is a full-length form sequence).

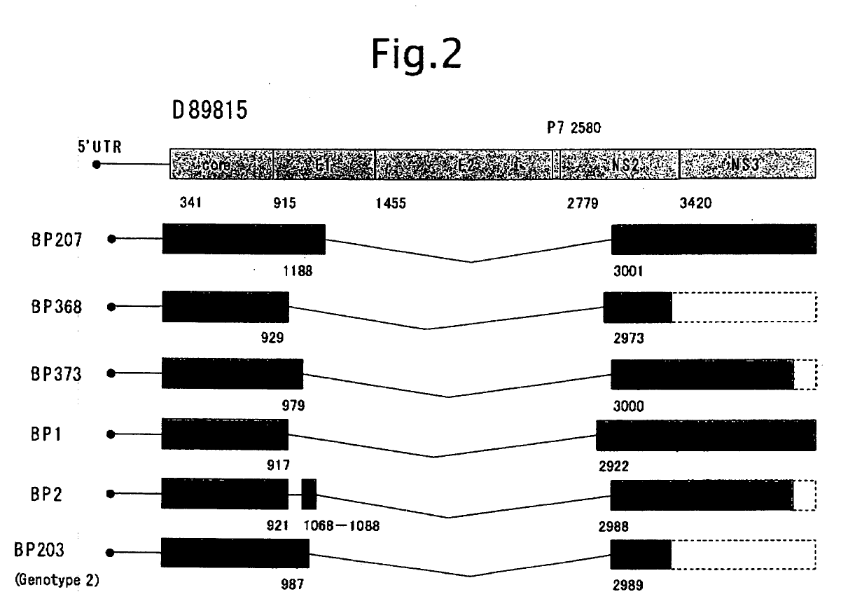

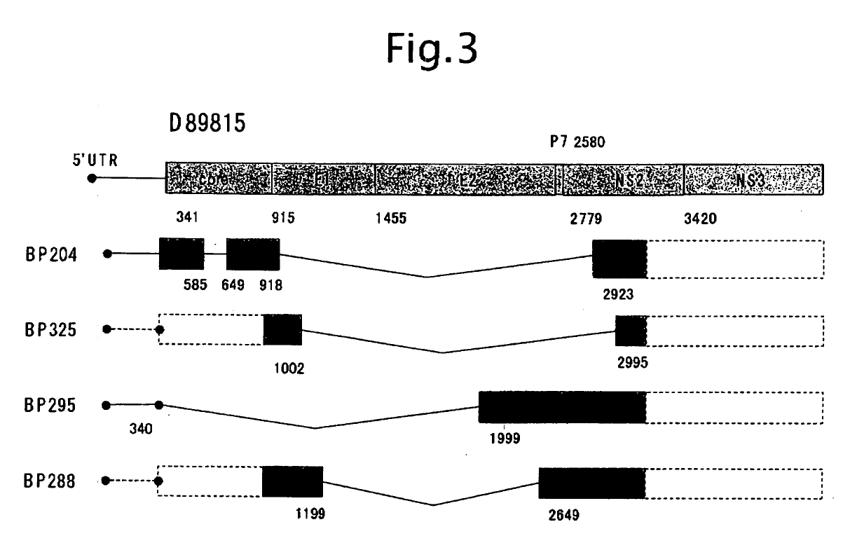

This TF genome is characterized in that a region encoding part or all of the core protein, the E1 protein and the E2 protein has been deleted. Specifically, as described in FIG. 2 and FIG. 3, it is characterized in that it does not retain the gene encoding the E1 and the E2 region in its complete form. In other words, it is a gene characterized in that the gene encoding part or all of the E1 protein and the E2 protein has been deleted. In the TF genome obtained at present, no TF genome that retains from position 1200 encoding E1 to position 1998 encoding E2 has been confirmed. The contiguous deletion of each TF gene is at least 63 bases, and at most 2043 bases. Also the total of deletions of one TF gene is 1449-2067 bases.

The most typical structure of the TF genome is a structure in which an about 2 kb gene encoding the E1 protein to the NS2 protein has been deleted (FIG. 2).

Also desirable are a gene having the above deletion and retaining 5′UTR, a gene encoding proteins downstream from NS3 and a gene retaining 3′UTR.

The TF gene is characterized by the structure of its deletion region. Thus, all nucleic acids are included in the present invention as long as they are the gene sequence of HCV. In addition to the genotype 1, the present invention was confirmed to have the TF gene of the genotype 2, and the TF genes with a sequence of genotypes 3 to 6 are also included.

The TF genome is autonomously replicating in the liver of patients with chronic active hepatitis C. There are also samples in the tissue of the patient in which the TF genome alone is amplified by PCR. This suggests that the TF genome is being preferentially replicated in the liver tissue instead of the FLF genome.

Thus, this TF genome can be used as a replicable replicon in the liver cells or the liver-derived cells. As the TF per se has an autonomous replicating ability, the structure as it is functions as a replicon. Also, in order to select a replicon, a drug resistance gene can be connected thereto and be selected with the drug. The drug resistance gene, such as neomycin etc. can be used. The TF genome that can be used as a replicon contains all of the above TF genome in which the structural region is deleted. Also, in addition, any structure of a TF genome having in-frame deletion of the HCV gene replicating in the liver can be used.

The most preferred replicon of the present invention is a replicon that has the IRES of the 5′-untranslated region, the core region, part of the E1 region, and a region encoding downstream from part of NS2 to the nonstructural protein, and 3′UTR of HCV, and a replicon that has the same structure as the TF genome replicating in vivo. Furthermore, those in which part of the replicon such as one having integrated therein the neomycin-resistant gene in the core region can also be used as the replicon.

Furthermore, the present invention provides a cell in which this replicon is replicated. HCV RNA having the structure of the TF genome is actually being replicated in the liver cell, and the replication system of the TF genome replicon using this structure is thought to reflect a virus replication system in the liver. The cell in which the replicon is replicated may be a subcultured cell derived from the liver or a primary hepatic cell. Cells not derived from the liver can also be used as long as the replicon is replicated therein. Cells in which the replicon is replicated include cells in which replicon is permanently replicated. Also, cells in which replicon is transiently replicated are included.

The replication system of the TF genome replicon is useful in screening drugs against HCV infection. This is because the TF genome is actually being replicated in the liver tissue. Using the replicon replication system in which this replicon is actually replicating, drugs that suppress HCV propagation can be screened. Also included are methods of evaluating drug efficacy using a replicon-replicating cell. Since this screening system targets the replicon of the TF genome being actually replicated in the liver, screening of more effective drugs can be expected. It is also useful as a method for evaluating the effect of screened drugs. In this case, it is important to carry out the evaluation of drugs with this method. If it is essential for the control of drugs, it can also be used as a method for producing drugs.

Furthermore, the present invention provides a method of detecting the TF genome. The TF genome has a deleted region. Any method that detects a gene having this deletion can be used. For example, a primer is designed outside to the 3′-end of the deleted region, and cDNA is synthesized from RNA. Primers are designed outside to the 5′ and 3′ so as to sandwich the deleted region of the cDNA, and by performing PCR, genes having deletion shorter than the FLF genome can be detected. After performing PCR, it is also possible to detect shorter TF genomes by Northern blotting.

This method of detecting the TF genome includes all the methods of detecting gene deletions in addition to the above-mentioned methods.

Also, by subtracting the amount of the FLF genome from the amount of the full-length FLF genome and the TF genome, it is also possible to determine the amount of the TF genome.

Primers are designed at the common region in the FLF genome and the TF genome having no deletions, and the amount of both genes is determined by RT-PCR. Then, by designing a PCR primer in the deleted region of the TF genome and determining the amount of the gene, the amount of the FLF genome alone can be determined. By comparing the amount of both the FLF genome and the TF genome and the amount of the FLF genome alone, the amount of the TF genome can be determined.

A primers for determining the amount of the FLF genome and the TF genome may be designed at any region as long as the FLF genome and the TF genome are overlapping therein. For example, a primer can be designed at 5′UTR or a region encoding nonstructural proteins NS3 and after, or at 3′UTR. Also, a primer for determining the FLF genome alone can be designed at any deleted region in the TF genome, for example, a region encoding structural proteins such as the gene region of core, E1 and E2, or a region of P7 or NS2 in which the deletion is observed. As a typical TF genome has a deletion of a nucleic acid sequence No. 1189 to 2922, the FLF genome alone can be detected by designing a primer at this region. More preferably, as all TF genomes obtained in Examples of the present invention have a deletion of 1200 to 1998, the FLF genome alone can be detected by designing a primer at this region.

The quantitation of HCV RNA gene can be carried out by the RT-PCR method. For example, competitive RT-PCR, the realtime PCR method or the like can be used.

The present invention provides an invention that relates to a polyprotein produced by the TF genome. The TF genome has an in-frame deletion and encodes a polyprotein. This polyprotein is one that does not have a polypeptide encoded in the deleted region compared to the polyprotein encoded in FLF genome. Also, it is a novel protein different from the normal polyprotein produced from the FLF genome, in which protein at the N terminal end upstream from the deleted region and a protein at the C terminal end downstream from the deleted region are fused.

The present invention also provides a protein that was processed from the above polyprotein. The above polyprotein is cleaved with a peptidase, and processing yields novel proteins such as a fusion protein of E1 and NS2, a fusion protein of E1 and E2 and a fusion protein of core and E2. The above polyprotein or processed protein is not produced from the FLF genome, and thus by detecting this protein, an effect similar to the detection of the TF genome can be obtained.

The present invention also provides a method of diagnosing the conditions of hepatitis by detecting or quantitating the TF genome or a polyprotein produced from the TF genome.

The present invention also provides an antibody specific to a polyprotein or a fusion protein produced from the TF genome. These antibodies are useful for detecting a polyprotein or a fusion protein.

The present invention also provides particles having a virus-like structure containing the TF genome. The TF genome is detected from the blood other than the liver cells. In the blood, the TF genome exists in association with the core protein. The virus particles or virus-like particles can also be used for diagnosis, treatment or the like.

### EXAMPLES

### Example 1

### Isolation and Analysis of a Truncated Genome Sequence

A patient liver section BP207 (0.5 mm×1 mm) was disrupted in 100 μl of the RIPA buffer (20 mM Tris-HCl [pH 7.5], 150 mM NaCl, 1% NP40, 0.1% deoxycholate, complete protease inhibitor cocktail [Roche Diagnostics Corporation]), and after centrifuging at 10 krpm for 5 minutes, the supernatant was collected. From this extract, using the High Pure Viral Nucleic Acid Kit (Roche Diagnostics Corporation), a nucleic acid was purified according to the manufacturer's instructions. To the nucleic acid purified, the HC9405R-1b primer was added, and using the MMLV reverse transcriptase (Invitrogen) a reverse transcription reaction was carried out at 42° C. for 1 hour according to the manufacturer's instructions to obtain cDNA.

To this reaction mixture, RNaseH (Invitrogen) was added, and reacted at 37° C. for 30 minutes to digest RNA. Using a portion of this reaction mixture, and using KlenTaq LA DNA polymerase (Clontech, BD Bioscience) in the presence of the HC-Long A1 primer and the T7-HC9313R primer, a polymerase chain reaction (PCR) was carried out at a thermal cycle reaction for 30 cycles with each cycle comprising 94° C. for 20 seconds and 68° C. for 9 minutes to amplify cDNA of HCV genomic RNA (HCV cDNA). Using a portion of this reaction mixture, PCR was further carried out in the presence of HC85F and HC9302R primers to amplify HCV cDNA.

The amplified fragment was separated on a 0.7% agarose gel electrophoresis, and using the QIAquick gel purification kit (QIAGEN), a DNA fragment was purified from the agarose gel according to the manufacturer's instructions. The purified SCV cDNA fragment was cloned into the PGEM-T easy vector (Promega) according to the manufacturer's instructions to transform the DH5α strain. A transformant that is ampicillin-resistant and that forms a white colony on an agar medium to which IPTG and x-gal had been added in a plate culture was selected, and cultured in a 2YT medium to which ampicillin had been added to 100 μg/ml. From the cultured cells, plasmid was purified using the Wizard Plus SV Miniprep DNA Purification system.

The sequence of HCV cDNA that has been cloned into the purified plasmid was analyzed using primers prepared as appropriate so as to be consistent with the vector and HCV cDNA by carrying out a reaction with the CEQ DTCS Quick Start Kit (Beckman Coulter) according to the manufacturer's instructions and analyzing with the CEQ2000 XL DNA analysis system (Software version 4.0.0, Beckman Coulter). Based on the data obtained, the sequence data was assembled and analyzed using the Sequencer (Version 4.1.2, Gene Codes Corporation) to determine the base sequence of HCV cDNA. The sequences of three types of HCV cDNA of clones LV207-0193-1, LV207-0193-6 and LV207-0193-15 were determined.

By comparing with the published sequence (D89851) of HCV cDNA, the isolated sequences were found to contain a sequence of positions 85 to 9302 which is a primer region used for PCR, but all clones lacked a sequence corresponding to positions 1189 to 3000. The predicted amino acid sequence was found that the sequence of LV207-0193-1 encodes a stretch of an amino acid length but lacks a sequence from the middle of E1 to the middle of NS2 without deviation from (in-frame with) the translation frame. Based on the determined sequence, a common sequence and a consensus sequence were determined using the MacVector (version 7), and by combining the suitable fragments of HCV cDNA clones obtained, HCV cDNA fragments having the consensus sequence were constructed.

Specifically, to the fragment of clone 1, a ClaI site at position 709 of SEQ ID NO; 1 was mutation-introduced using Cla_s and Cla_as primers and the Quick Mutagenesis Kit (Stratagene) according to the manufacturer's instructions. From LV207-0193-1, a fragment having from the ClaI site at position 709 to the AfeI site at position 1063, a fragment from the HpaI site at 4169 to the SacI site at 5569, and a fragment having from the SfiI site at 6687 to the BglII site at 7123 were isolated and used for construction. And from LV207-0193-6, a fragment having from the AfeI site at 1063 to the BsiWI site at 1265 was isolated and used.

From LV207-0193-15, a fragment having from the BsiWI site at position 1265 to the HpaI site at position 4169, a fragment having from the SacI site at 5569 to the SfiI site at 6687, and a fragment having from the BglII site at 7123 to the HindIII site at 7386 were isolated and used for construction. By combining these fragments, and further combining a fragment of HindIII at 7383 to XbaI at 7786 containing 3′UTR isolated from a patient tissue, they were inserted into the ClaI site to the XbaI site of a cloning vector pBluescript SKII(−) (Stratagene) to construct pLVC_ClaXba7.2K having a sequence from positions 709 to 7786 of SEQ ID NO: 1. The restriction enzymes and T4 DNA ligase were purchased from New England Biolabs, Takara Shuzo, Toyobo and NIPPON GENE Co., Ltd.

On the other hand, the end of HCV cDNA was isolated as follows. The cDNA reaction mixture treated with the above RNaseH was subjected to PCR comprising 35 thermal cycles of 94° C. for 20 seconds, 55° C. for 30 seconds and 72° C. for 1 minute using HCLongH1 and HC705R and the JumpStart RedTaq DNA polymerase (Sigma) to amplify a fragment corresponding to positions 1 to 709 of the previously reported HCV cDNA. The cloning and analysis of fragments were carried out according to the standard methods. As a result, HCV cDNA, pLV207-0007, containing positions 1 to 709 of SEQ ID NO: 1 was obtained. By PCR using this as the template and the T7-HIV2 primer and the CoreCla_as primer, an about 0.7 kb fragment was amplified, and by cloning to pGEM-T Easy, pT7_LV207_{—}0007 was obtained.

An about 0.7 kb fragment obtained by cleaving pT7_LV207_{—}0007 with NotI and ClaI and an about 7.2 kb fragment obtained by cleaving pLVC_ClaXba7.2K with ClaI and XbaI were inserted in between the NotI site and the XbaI site of pcDNA3.1(+) (Invitrogen) to obtain a plasmid pcD-LV207TF having inserted therein HCV cDNA having a sequence from positions 1 to 7786 of SEQ ID NO: 1.

Separation of the 3′UTR Gene from the Patient Tissue

In a method similar to the above, RNA was recovered from the tissue of a patient with chronic hepatitis. To 2.5 μl of RNA, 5 pmole (0.5 μl) of primer 8913F was added, incubated at 70° C. for 3 minutes, and quickly cooled on ice. To this, 2 μl of 5× First-Strand Buffer, 1 μl of 0.1 M DTT, 0.5 μl of 20 mM dNTP, 20 units of RNase Inhibitor (TAKARA), and 0.5 μl of the MMLV reverse transcriptase were added, and a diethyl pyrocarbonate-treated sterile water was added thereto to a total volume of 10 μl. The mixture was reacted at 42° C. for 60 minutes. In order to disrupt RNA, 12 U of RNaseH (TAKARA, 60 U/μl) was added, incubated at 37° C. for 30 minutes, and then incubated at 72° C. for 3 minutes for the inactivation of RNaseH, and cDNA was used.

Using primers 8913F and RP2, 2 μl of this cDNA was subjected to PCR in a method similar to the above. The PCR product was subjected to the second PCR with primers 8939F and R1 to obtain a PCR product of about 600 bases. The PCR product was cloned into the pGEM-T Easy vector, and the nucleic acid sequence was determined in a method described above.

The following is part of primer sequences used in cloning and the construction of genes:

| | |
|---|---|
| 1b160Bam: | |
| 5′-cgcggatcct tagtcctcca | (SEQ ID NO: 49) |
| gaacccggac ac-3′ | |
| chiba-as: | |
| 5′-tgcacggtct acgagacct-3′ | (SEQ ID NO: 50) |
| chiba-s: | |
| 5′-tagtggtctg cggaaccggt-3′ | (SEQ ID NO: 51) |
| core_cla_as: | |
| 5′-gccgcatgta agggtatcga tgacc-3′ | (SEQ ID NO: 52) |
| core_cla_s: | |
| 5′-ggtcatcgat acccttacat gcggc-3′ | (SEQ ID NO: 53) |
| eco_npt_as: | |
| 5′-gcgaattctt atcagaagaa | (SEQ ID NO: 54) |
| ctcgtcaaga ag-3′ | |
| HC1b9405R: | |
| 5′-gcctattggc ctggagtgtt tagctc-3′ | (SEQ ID NO: 55) |
| HC85F: | |
| 5′-atggcgttag tatgagtgtc | (SEQ ID NO: 56) |
| gtgcagcct-3′ | |
| HC705R: | |
| 5′-agccgcatgt aagggtatcg atgac-3′ | (SEQ ID NO: 57) |
| HC1986S: | |
| 5′-tggttcggct gyacatggat gaa-3′ | (SEQ ID NO: 58) |
| HC2199AS: | |
| 5′-ggrtagtgcc aragcctgta tgggta-3′ | (SEQ ID NO: 59) |
| HC9302R: | |
| 5′-tcgggcacga gacaggctgt | (SEQ ID NO: 60) |
| gatatatgtc t-3′ | |
| HClongA1: | |
| 5′-atcgtcttca cgcagaaagc | (SEQ ID NO: 61) |
| gtctagccat-3′ | |
| HClongH1: | |
| 5′-gccagccccc tgatgggggc | (SEQ ID NO: 62) |
| gacactccac c-3′ | |
| Nde_core9_as: | |
| 5′-aatcatatgt ctttgaggtt | (SEQ ID NO: 63) |
| taggatttgt-3′ | |
| Nde_npt_s: | |
| 5′-gacatatgat tgaacaagat | (SEQ ID NO: 64) |
| ggattgcac-3′ | |
| SbfH1: | |
| 5′-gtcctgcagg ccagccccct | (SEQ ID NO: 65) |
| gatgggggcg aca-3′ | |
| SbfNpt-R: | |
| 5′-gacctgcagg ttatcagaag | (SEQ ID NO: 66) |
| aactcgtcaa gaag-3′ | |
| T7_H1V2: | |
| 5′-gccttaatta atacgactca | (SEQ ID NO: 67) |
| ctataggcca | |
| gccccctgatgggggcgaca-3′ | |
| T7_HclongH1: | |
| 5′-tctagtcgac ggccagtgaa | (SEQ ID NO: 68) |
| ttgtaatacg actcactata gggcggccag | |
| ccccctgatgggggcgacac tccacc-3′ | |
| T7_HC9313b: | |
| 5′-tctagtcgac ggccagtgaa | (SEQ ID NO: 83) |
| ttgtaatacg actcactcta | |
| gggcggcggg gtcgggcwcg | |
| ngacabgctg tga-3′ | |

### Example 2

### Isolation and Analysis of cDNA to TF HCV Genomic RNA from a Patient

Subsequently, the detection of TF HCV-RNA from 23 samples of liver biopsy from the liver of chronic active hepatitis and 3 samples of BP1, BP2 and BP3 that are obtained from surgically removed liver tissues from patients with hepatic carcinoma.

Extraction of RNA was attempted.

### RNA Extraction

According to a protocol of “Isolation of RNA from trace samples” of the extraction reagent for RNA, ISOGEN (NIPPON GENE Co., Ltd.), RNA was extracted. To a liver section of about 0.5 mm×1 mm in dimension from a patient, 0.8 ml of ISOGEN was added, the tissue section was loosened with a 1 ml pipette tip, and disrupted by pipetting. After standing at room temperature for 5 minutes, 0.2 ml of chloroform was added, and after vigorous shaking for 30 seconds, it was allowed to stand at 4° C. for 5 minutes. After centrifuging at 12,000 g, 4° C. for 15 minutes in a refrigerated micro centrifuge, the aqueous phase was collected.

About 5 μl of yeast tRNA was added, 0.8 ml of isopropanol was added, and the mixture was allowed to stand at 4° C. for 30 minutes or overnight. After centrifuging at 12,000 g, 4° C. for 15 minutes, the supernatant was discarded, and the pelleted RNA was collected. To the pellet was added 1 ml of 70% ethanol, which was vigorously shaken and then centrifuged at 12,000 g, 4° C. for 15 minutes. The supernatant was discarded, and the same procedure was repeated twice to wash the pellet. After the last washing, the pellet was air-dried for 10 minutes, dissolved in 50 μl of a diethyl pyrocarbonate-treated sterile water to prepare a RNA sample. The sample was stored at −80° C. until use.

Synthesis of cDNA

The deleted region of the TF genome obtained from the tissue of BP207 for which cDNA was synthesized from the RNA sample extracted from the liver biopsy tissue was nucleic acids Nos. 1189 to 3000 of the HCV gene, several primers for cDNA synthesis were designed at the 3′-untranslated region side from 3000 and cDNA was synthesized. The reverse transcriptase used was the MMLV reverse transcriptase from GIBCO-BRL, and synthesis was carried out according to the attached protocol.

To 2.5 μl of RNA, 5 pmole (0.5 μl) of primers was added, incubated at 70° C. for 3 minutes, and quickly cooled on ice. To this, 2 μl of 5× First-Strand Buffer, 1 μl of 0.1 M DTT, 0.5 μl of 20 mM dNTP, 20 units of RNase Inhibitor (TAKARA), and 0.5 μl of the MMLV reverse transcriptase were added, and a diethyl pyrocarbonate-treated sterile water was added thereto to a total volume of 10 μl. The mixture was reacted at 42° C. for 60 minutes. In order to disrupt RNA, 12 U of RNaseH (TAKARA, 60 U/μl) was added, incubated at 37° C. for 30 minutes, and then incubated at 72° C. for 3 minutes for inactivation of RNaseH, and cDNA was used.

Primers used for cDNA synthesis were any of the following:

(SEQ ID NO: 69)

| | |
|---|---|
| 5035R: | 5′-AGGCCTGTGA AGACGCTCTC CCAGAACT-3′ |

(SEQ ID NO: 70)

| | |
|---|---|
| HC3297R: | 5′-GGTGATGAC CTTGGTCTCC AT-3′ |

(SEQ ID NO: 71)

| | |
|---|---|
| HC3481R: | 5′-GCTTAGAGGC TAGTGATGAT GCAACCAAGT AC-3′ |

(SEQ ID NO: 72)

| | |
|---|---|
| HC3945R: | 5′-GGCGACCGCA TAGTAGTTTC CATA-3′. |

The primers used are described in Table 1.

### Amplification by Polymerase Chain Reaction (PCR)

Using cDNA, PCR was carried out with a combination of several primers from 5′UTR to the region encoding NS3. For PCR, TaKaRa LA Taq (TAKARA) was used and reacted according to the attached protocol. Two μl of cDNA, 2.5 μl of 10×LA PCR buffer II (Mg²⁺ free), 2.5 μl of 25 mM MgCl₂, 2.5 μl of 2.5 mM dNTP, 10 pmole of a sense primer, 10 pmole of an antisense primer, 0.25 μl (5 units/μl) of TaKaRa LA Taq were added, to which a sterile water was added to 25 μl, and PCR was carried out. For the reaction, the master cycler gradient (Eppendorf Iatron) was used. The reaction profile comprised, after heating at 94° C. for 2 minutes, 10 cycles of denaturation at 94° C. for 20 seconds, annealing at 62° C. for 30 seconds and extension at 68° C. for 3 minutes, and then 25 cycles of denaturation at 94° C. for 20 seconds, annealing at 58° C. for 30 seconds and extension at 68° C. for 3 minutes.

Using 2.5 μl of the product from the PCR (1st PCR), the second-round PCR (2nd PCR) was carried out. 2.5 μl of 1st PCR product, 2.5 μl of 10×LA PCR buffer II (Mg²⁺ free), 2.5 μl of 25 mM MgCl₂, 2.5 μl of 2.5 mM dNTP, 10 pmole of a sense primer, 10 pmole of an antisense primer, and 0.25 μl (5 units/μl) of TaKaRa LA Taq were added, to which a sterile water was added to 25 μl, and PCR was carried out in a reaction profile similar to the first PCR.

Primers used for cDNA synthesis were combinations of any of the following:

(SEQ ID NO: 56)

| | |
|---|---|
| HC85F: | 5′-ATGGCGTTAG TATGAGTGTC GTGCAGCCT-3′ |

(SEQ ID NO: 73)

| | |
|---|---|
| HC813S: | 5′-CTGGAGGACG GCGTGAACTA TGCAACAGGG AA-3′ |

(SEQ ID NO: 74)

| | |
|---|---|
| HC841S: | 5′-GGAACTTGCC CGGTTGCTCT TTCTCTATCT TC-3′ |

(SEQ ID NO: 75)

| | |
|---|---|
| HC3206R: | 5′-TGGGGCAAGA TGGTTATAAA C-3′ |

(SEQ ID NO: 76)

| | |
|---|---|
| HC3174AS: | 5′-GGGGTAAGAT GGTTATAAAC GTACGTACCT G-3′ |

(SEQ ID NO: 77)

| | |
|---|---|
| HC3111AS: | 5′-ATAATGACCC CCGGCGACTT TCCGCACTAA C-3′ |

(SEQ ID NO: 70)

| | |
|---|---|
| HC3297R: | 5′-GGTGATGAC CTTGGTCTCC AT-3′ |

(SEQ ID NO: 71)

| | |
|---|---|
| HC3481R: | 5′-GCTTAGAGGC TAGTGATGAT GCAACCAAGT AC-3′ |

(SEQ ID NO: 78)

| | |
|---|---|
| HC3759R: | 5′-TGACATCAGC ATGTCTCGTG ACCA-3′ |

(SEQ ID NO: 61)

| | |
|---|---|
| HCLONGA1: | 5′-ATCGTCTTCA CGCAGAAAGC GTCTAGCCAT-3′ |

(SEQ ID NO: 72)

| | |
|---|---|
| HC3945R: | 5′-GGCGACCGCA TAGTAGTTTC CATA-3′ |

The combinations of 1st and 2nd primers used are described in Table 1.
Analysis of PCR products

PCR products after the completion of reaction were analyzed by electrophoresis. Using 1% agarose gel, samples were subjected to submerged electrophoresis in 1×TAE buffer (Tris-HCl 40 mM, glacial acetic acid 40 mM, EDTA 1 mM). A gel loading buffer was added to the PCR product, and after electrophoresis, stained with ethidium bromide and the PCR product was observed under UV.

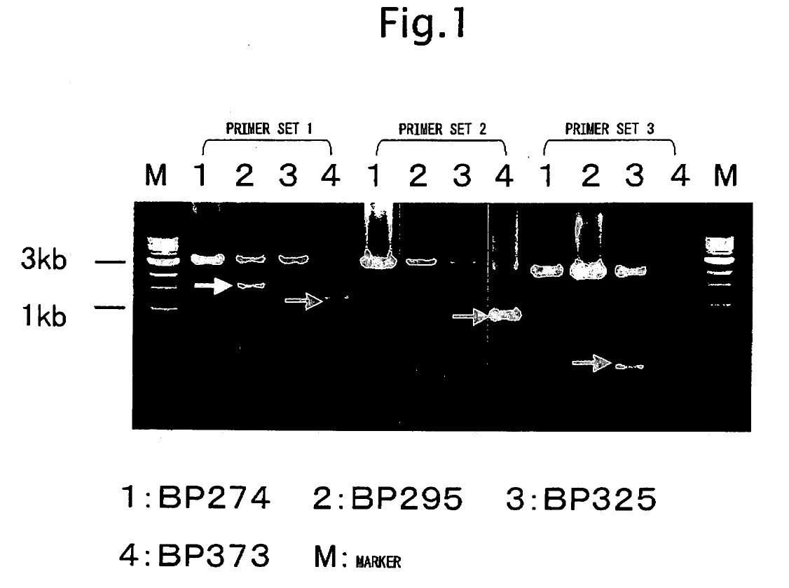

The image of electrophoresis of the HCV gene amplified with three sets of primers from 4 samples of RNA is shown in FIG. 1. With the combination of HC85F and HC3297R, 841S and 3759R, and 841S and 3111AS as the primers of the 2nd PCR, FLF HCV-RNA of an about 3.1 kb, 2.9 kb, and 2.2 kb should be detected, respectively. For the sample (BP274) of number 1, a PCR product of the FLF was only found with any combination of primers. For the sample (BP295) of number 2, with the combination of HC85F and HC3297R, about 3.1 kb FLF and 2 kb TF, with the combination of 841S and 3759R, an about 2.9 kb FLF, and with the combination of 841S and 3111AS, an about 2.2 kb FLF were detected.

For the sample (BP325) of number 3, with the combination of HC85F and HC3297R, an about 3.1 kb FLF, with the combination of 841S and 3759R, an about 2.9 kb FLF, and with the combination of 841S and 3111AS, an about 2.2 kb FLF and 300 bp TF were detected. For the sample (BP373) of number 4, with the combination of HC85F and HC3297R, an about 1.2 kb TF, with the combination of 841S and 3759R, an about 1 kb TF were detected, and with the combination of 841S and 3111AS, no PCR product was detected. There were samples in which FLF was only detected, samples in which FLF and TF were detected, and samples in which TF was only detected, though this varies with the combination of primers. For 26 samples, PCR products obtained with each combination of cDNA synthesis primer, the 1st PCR and the 2nd PCR primers are shown in Table 1.

TABLE 1

| | | | 3945R | 3481R | 3945R | 3945R | 3297R | 3174R | S035R | | |
|---|---|---|---|---|---|---|---|---|---|---|---|
| | | cDNA synthesis | HClongA1- | HClongA1- | 813S- | 813S- | HClongA- | 813S- | HClongA1- | | Amount of |
| | | primer | 3945R | 3481R | 3945R | 3174AS | 3297R | 3174AS | 5035R | | HCV-RNA |
| | Sample | 1st PCR primer | 85F- | 85F- | 841S- | 841S- | 85F- | 841- | 85F- | Geno- | (copis/ul |
| | No. | 2nd PCR primer | 3297R | 3297R | 3759R | 3111AS | 3174AS | 3111AS | 3945R | type | RNA) |
| 1 | BP171 | | — | 1.5k | 3k | 2.3k + | 1.8k | NT | NT | 1 | 424.2 |
| | | | | | | 0.5k | | | | | |
| 2 | BP178 | | — | — | — | — | — | NT | NT | 2 | 28.9 |
| 3 | BP193 | | 3k | 3k | 3k + 1k | 2.3k | 3k + | NT | NT | 1 | 1853.0 |
| | | | | | | | 0.5k | | | | |
| 4 | BP201 | | — | — | — | — | — | NT | NT | ? | 35.0 |
| 5 | BP203 | | — | 1.2k(TF) | — | — | 1.1k(TF) | NT | NT | 2 | 467.4 |
| 6 | BP204 | | 3k(TF^{#})* | 1k | NT | NT | NT | NT | NT | 1 | NT |
| 7 | BP235 | | — | — | 3k | — | — | NT | NT | 2 | 1009.0 |
| 8 | BP245 | | 1.2k | — | NT | NT | NT | NT | NT | 1 | |
| 9 | BP248 | | — | — | — | — | 3k | NT | NT | 2 | 131.1 |
| 10 | BP257 | | — | — | — | 2.3k | — | NT | NT | 1 | 115.9 |
| 11 | BP274 | | 3k | 3k | 3k | 2.3k | 1k | NT | NT | 1 | 1375.0 |
| 12 | BP288 | | 3k | 3k | 3k | 2.3k + | 3k + | NT | NT | 1 | 388.6 |
| | | | | | | 0.8k(TF) | 1.5k | | | | |
| 13 | BP295 | | 3k | 3k + | 3k | 2.3k | 3k | NT | NT | 1 | 65.6 |
| | | | | 1.5k(TF) | | | | | | | |
| 14 | BP297 | | — | — | — | — | — | NT | NT | 2 | 330.6 |
| 15 | BP298 | | — | 3k + | — | — | — | KT | NT | ? | 1582.0 |
| | | | | 1.7k + | | | | | | | |
| | | | | 0.5k | | | | | | | |
| 16 | BP299 | | — | — | 3k | 2.3k | 0.3k | NT | NT | 1 | 151.8 |
| 17 | BP305 | | — | 3k + | — | — | — | NT | NT | 2 | 203.0 |
| | | | | 1.7k + | | | | | | | |
| | | | | 0.5k | | | | | | | |
| 18 | BP325 | | 3k | 3k | 3k | 2.3k + | 0.8 + | NT | NT | 1 | 931.1 |
| | | | | | | 0.3k(TF) | 0.5k | | | | |
| 19 | BP331 | | 3k | 1.5k(TF) + | 3k | 2.3k + | 1.3k | NT | NT | 1 | 627.0 |
| | | | | 0.5k | | 1.2k | | | | | |
| 20 | BP357 | | — | — | — | — | — | NT | NT | 2 | 710.4 |
| 21 | BP368 | | — | 1k(TF) | NT | NT | — | NT | NT | 1 | NT |
| 22 | BP372 | | 3k | 3k | 3k | 2.3k | 2.5k | NT | NT | 1 | 916.9 |
| 23 | BP373 | | 1.3k(TF) | — | 1k(TF) | — | 1.2k | NT | NT | — | 7979.0 |
| 24 | BP1 | | 1.3k(TF) | NT | 1k(TF) | NT | NT | 0.3k(TF) | 1.9k(TF) | NT | 442.0 |
| 25 | BP2 | | 1.3k(TF) | NT | 1k(TF) | NT | NT | NT | NT | NT | 88.8 |
| 26 | BP3 | | — | NT | — | NT | NT | NT | NT | NT | 46.7 |

*Bold letters show the TF genome of which nucleic acid sequence has been determined and described in the sequence list.
^{#}TF means that deletion was confirmed.
NT: not tested

### Sequence Determination

The obtained PCR products thought to be FLF and TF were cloned into plasmids and the sequences were determined.

A PCR product was excised from the electrophoresed agarose gel, purified by the QIAquick PCR Purification Kit (QIAGEN), and extracted into 40 μl of sterile water. To 5 μl of DNA and 0.5 μl of pGEM-T Easy Vector (Promega), 1 μl of 10× T4 ligase buffer, 1 μl of T4 DNA ligase and 2.5 μl of sterile water were added, and reacted at 16° C. for 1 hour to ligate the DNA and the vector. To competent cells of E. coli DH5α prepared by the method of Inoue et al. (Gene, vol. 96, 1990, pp. 23-28), DNA was added, and transformed according to a standard method.

The colonies that developed were cultured for one night and day in a 2×YT medium, miniprepped by the Wizard Plus SV Minipreps DNA Purification System (Promega), and plasmid DNA was collected. The plasmid DNA in which a PCR product has been inserted was analyzed and sequenced by the CEQ 2000XL DNA analysis system (BECKMAN COULTER). It was reacted using the CEQ2000 Dye Terminator Cycle Sequencing with Quick Start Kit according to the attached protocol and analyzed. As the sequence primers, primers of pGEM-T Easy and those suitable for the HCV sequence were selected as appropriate. The sequence was analyzed by the MacVector (Accelrys) and Sequencher (Gene Codes Corporation).

The nucleic acid sequence of the detected TF genome and the amino acid sequences predicted therefrom are shown in the sequence list. Sample BP203 are shown in SEQ ID NOs: 9-12, BP204 in SEQ ID NOs: 13 and 14, BP208 in SEQ ID NOs: 15 and 16, BP295 in SEQ ID NOs: 17 and 18, BP325 in SEQ ID NOs: 19 and 20, BP368 in SEQ ID NOs: 21 and 22, BP373 in SEQ ID NOs: 23 to 28, BP1 in SEQ ID NOs: 29 to 34, and BP2 in SEQ ID NOs: 35 to 38. Those in which FLF was detected and those in which TF was detected with any combination of primers are summarized in Table 2.

TABLE 2

| | TF | FLF | Genotype |
|---|---|---|---|
| BP207 | ◯ | | 1 |
| BP203 | ◯ | | 2 |
| BP368 | ◯ | | 1 |
| BP373 | ◯ | | 1 |
| BP1 | ◯ | | 1 |
| BP2 | ◯ | | 1 |
| BP204 | ◯ | ◯ | 1 |
| BP288 | ◯ | ◯ | 1 |
| BP295 | ◯ | ◯ | 1 |
| BP325 | ◯ | ◯ | 1 |
| BP331 | ◯ | ◯ | 1 |
| BP171 | | ◯ | 1 |
| BP193 | | ◯ | 1 |
| BP257 | | ◯ | 1 |
| BP274 | | ◯ | 1 |
| BP299 | | ◯ | 1 |
| BP372 | | ◯ | 1 |

In 6 samples TF alone was detected, in 5 samples both FLF and TF were detected, in 6 samples FLF alone was detected, and in the rest of samples neither was detected. Most of the samples in which neither was detected were genotype 2. Since the cDNA synthesis primer, and 1st PCR and the 2nd PCR primers were synthesized based on the sequence of genotype 1, the sequences may not correspond to the sequence of HCV-RNA of genotype 2, and this may be a reason that PCR products were not obtained in many samples.

### Analysis of the TF Genome

The sequence of TF HCV-RNA was compared to the sequence of HCV-RNA of No. D89815. FIG. 2 shows the structures of BP207, BP368, BP373, BP1, BP2 and BP203 in which TF alone was detected. Though the deleted region was about 2 kb, none had the deletion of the same region as BP207, and regions deleted in each patient were different. BP203, the only sample among the samples of genotype 2 in which TF was detected, had a similar deletion, and the deleted region was present at nucleic acid Nos. 988 to 2988.

Common in these HCV-RNAs is the occurrence of in-frame deletion as in BP207, and it is thought that HCV polyprotein is being synthesized at regions excluding the deleted region. In particular, the gene encoding the core protein is retained in the entire form and the core protein per se is normally synthesized, and none has the deletion of transmembrane domains at two sites at the C terminal end of NS2, and thus it is suggested that proteins at NS3 and after can be normally expressed. Furthermore, since the regions encoding the E1 and NS2 proteins are connected in-frame, it is thought that a fusion protein of E1 and NS2 is being produced.

Then, for the samples in which both types of TF and FLF were detected, the structures of the TF sequences were compared (FIG. 3). The sequences of BP204, BP325, BP295 and BP288 were confirmed. In BP204 and BP295 in which the gene corresponding to the region encoding the core protein has been obtained, unlike the sample in which TF alone was detected, part or all of the core region was deleted. Also, in some samples, NS2 was retained at the part subsequent to the deleted region and part of the E2 region was deleted.

However, HCV-RNA in these samples also had in-frame deletion as in BP207, and thus excluding the deleted region, it is thought that HCV polyprotein is being synthesized.

### Study on the HCV-RNA Sequence of TF and FLF

For sample of patients with chronic active hepatitis in which HCV-RNAs of TF and FLF were obtained, the nucleic acid sequences of the overlapping fragment and the predicted amino acid sequences were compared. A PCR product of the length of TF was cloned into a vector in a similar manner, and the nucleic acid sequence was determined. The sequences of FLF of BP204, BP325 and BP208 are shown in SEQ ID NOs: 39 and 40, 41 and 42, and 43 and 44, respectively. Comparison of the nucleic acid sequences and amino acid sequences revealed that it is 96.7% for nucleic acid and 97.5% for amino acid in BP325, and 97.3% for nucleic acid and 97.7% for amino acid in BP288, suggesting that they are viruses belonging to the same quasispecies. On the other hand, in BP204, sequences of TF and FLF were different with nucleic acid 82.6% and amino acid 83.6%.

TABLE 3

| | Nucleic acid | Amino acid |
|---|---|---|
| BP204 | 82.6% | 83.6% |
| | (298n.t.) | (98a.a.) |
| BP325 | 96.70% | 97.50% |
| | (245n.t.) | (81a.a.) |
| BP288 | 97.3% | 97.7% |
| | (786n.t.) | (264a.a.) |

Separation of TF-HCV-RNA from the Serum

For samples in which TF-HCV-RNA was detected from the liver tissue, separation of TF-HCV-RNA from the serum was attempted. It was extracted using the High Pure Viral RNA Kit (Roche) from the serum collected simultaneously with the liver biopsy. From 200 μl of the serum, it was extracted into 50 μl of the elution buffer according to the attached protocol. As used herein, serum corresponding to BP368, which is a sample for liver biopsy, is designated as S368.

Using 2.5 μl of RNA, cDNA was synthesized from RNA, and PCR products were obtained in a method similar to that in liver biopsy. When the 3297R primer was used in cDNA synthesis, HCLONGA1 and 3297R were used in the 1st PCR and 85F and 3174AS were used in the 2nd PCR for S368, a 1.2 kb possible PCR product of TF was obtained (Table 4).

TABLE 4

| | | | cDNA synthesis | | | | Amount of |
|---|---|---|---|---|---|---|---|
| | | | primer | 3945R | 3481R | 3297R | HCV-RNA |
| | | | 1st PCR primer | A1-3945R | A1-3481R | A1-3297R | (copis/ul |
| | | Genotype | 2nd PCR primer | 85F-3297R | 85F-3297R | 85F-3174AS | RNA) |
| 1 | S171 | 1 | | — | — | — | 12.3 |
| 3 | S193 | 1 | | — | — | — | 36.7 |
| 5 | S203 | 2 | | — | — | — | — |
| 6 | S204 | 1 | | — | — | — | 140.9 |
| 7 | S207 | 1 | | — | — | — | 713 |
| 12 | S288 | 1 | | — | — | — | — |
| 15 | S298 | ? | | — | — | — | 1.2 |
| 17 | S305 | 2 | | — | — | — | 4.2 |
| 18 | S325 | 1 | | — | — | — | 9.9 |
| 19 | S331 | 1 | | — | — | — | 3.1 |
| 21 | S368 | 1 | | — | — | 1.2 kbp(TF) | 297.6 |
| 23 | S373 | 1 | | — | — | — | 6.2 |

* Bold letters show the TF genome of which nucleic acid sequence has been determined and described in the sequence list.

Then, for S204, S207 and S368 which had a relatively large amount of RNA in the quantitation of HCV-RNA at the 5′UTR region, cDNA synthesis to PCR were carried out with different combinations of primers. As a result, a PCR product with the length of TF was obtained for s207 and S368 and a PCR product with the length of FLF was obtained for S204 (Table 5). The PCR products were similarly cloned into the pGEM-T Easy vector and the sequences were determined. The sequences determined for S207 and S368 are shown in SEQ ID NOs: 45 and 46 and SEQ ID NOs: 47 and 48, respectively. When the sequence homology of the nucleic acid sequence and the amino acid sequence for the overlapping region of BP207 and S207, and BP368 and S368 are compared, they were 99.4% and 99.3% for BP207 and S207, and 98.8% and 97.3% for P368 and S368, suggesting that they are viruses belonging to the same quasispecies (Table 6). This indicates that TF-HCV-RNA replicated in the liver is being released into the serum by some system.

TABLE 5

| | cDNA synthesis | 3945R | 3481R | 3297R | 3565AS | 3519AS | 3447AS |
|---|---|---|---|---|---|---|---|
| | 1st PCR | 813S-3297R | 813S-3297R | 813S-3297R | 813S-3297R | 813S-3297R | 813S-3297R |
| Sample No. | 2nd PCR | 841S-3174AS | 841S-3174AS | 841S-3174AS | 841S-3174AS | 841S-3174AS | 841S-3174AS |
| S204 | | — | — | — | — | 2.2 kbp(FLF) | — |
| S207 | | — | — | 0.5 kbp(TF) | — | — | 0.5 kbp |
| S368 | | 0.5 kbp(TF) | 0.5 kbp(TF) | 0.5 kbp(TF) | 0.5 kbp(TF) | 0.5 kbp(TF) | 0.5 kbp |

* Bold letters show the TF genome of which nucleic acid sequence has been determined and described in the sequence list.

TABLE 6

| | Nucleic acid | Amino acid |
|---|---|---|
| BP207 | 99.4% | 99.3% |
| vs S207 | (490n.t.) | (163a.a.) |
| BP368 | 98.8% | 97.3% |
| vs S368 | (1018n.t.) | (263a.a.) |

### Example 3

### Construction of HCV RNA Replicon

By inserting a linker fragment obtained by annealing the XhoX-Xba-s oligomer and the XhoX-Xba-as oligomer in between the XhoI site and the XbaI site of pBluescript IISK(+), pBSIISK(+) ΔXX was constructed. Also, by subjecting pLV207-0007 to PCR using the Sbf_H1 primer and the Cla_as primer, an about 0.7 kb fragment was amplified, which was cloned into pGEM-T Easy to obtain pLVC-0007Sbf. By linking and inserting an about 0.7 kb fragment obtained by cleaving pLVC-0007Sbf with NotI and ClaI and an about 7.2 kb fragment obtained by cleaving pLVC_ClaXba7.2K with ClaI and XbaI in between the NotI site and the XbaI site of pBSIISK(+)ΔXX, pSbf-LV207TF was obtained.

On the other hand, by adding the T7-HC9313b primer to RNA purified from HCV antibody-positive serum G14, cDNA was synthesized using the SuperscriptII reverse transcriptase (Invitrogen) according to the manufacturer's instructions. From this cDNA reaction mixture, HCV cDNA was amplified by PCR (35 cycles with one cycle comprising 95° C. for 30 seconds, 55° C. for 1 minute, 74° C. for 1 minute) using EX-Taq DNA polymerase (Takara Shuzo) in the presence of the T7-HClongHl primer and the lb160Bam primer. After separating fragments amplified by PCR on agarose gel electrophoresis, DNA was purified from the agarose gel using the QIAquick gel kit. The purified fragment was cloned into the pT7-blue T (Novagen), and the sequence was determined using the Applied Biosystems DNA sequencer 377A using reagents and conditions recommended by the manufacturer.

Then, HCV cDNA was amplified by PCR using the T7-HIV2 primer and the nde_core9_as primer. On the other hand, PCR was carried out with nde_npt_S and the EcoNpt_as primer as the template using pcDNA3.1(+), and a neomycin-resistant gene fragment was amplified. By linking these fragments with the restriction site of NdeI and then cloning this into pBluescriptIISK(−), a fusion fragment of 5′UTR, the first 9 amino acids of core of HCV cDNA and the neomycin-resistant gene (neomycin phosphotransferase, NPT-II) was constructed. A plasmid having this fragment was further subjected to PCR using the T7-H1V2 primer and the Sbf_Npt_R primer to prepare a fragment having a PacI site at the 5′-end and a SbfI site at the 3′-end. By cloning this into pGEM-T Easy, pG14UTRcNEO was obtained.

By inserting an about 1.2 kb fragment obtained by cleaving pG14UTRcNEO with NotI and SbfI into between the NotI site and the SbfI site of pSbf-LV207TF, pLV207TFRepG14 was obtained. Using this plasmid DNA cleaved with XbaI and NotI as the template, RNA was synthesized using the Megascript T7 kit (Ambion). RNA was purified according to the manufacturer's instructions.

Human hepatoma cells (Huh7, JCRB0403) were cultured at 5% carbon dioxide at 37° C. in the Dulbecco's Modified Eagle's Medium (D-MEM, IWAKI) supplemented with 10% fetal bovine serum (FBS), penicillin and streptomycin to 50 U/ml and 50 ug/ml, respectively. The subconfluent cells were treated with trypsin and EDTA to detach them from the culture plate, and resuspended into a serum-added medium to inactivate trypsin. After washing twice with PBS, they were suspended in Cytomix (120 mM potassium chloride, 10 mM potassium phosphate, 5 mM magnesium chloride, 25 mM HEPES, 0.15 mM calcium chloride, 2 mM EGTA, pH 7.6) supplemented 1.25% DMSO, it was then transferred to an electroporation cuvette with a gap of 0.4 cm.

After adding a suitable amount of RNA to cells, they are fully cooled on ice for 5 minutes. Cells were pulsed at 270 V and 960 μF using the electroporator (Bio-Rad). They are immediately resuspended into 8 ml of a medium, and are transferred into culture plates. After culturing for a certain period, the cells are detached with 0.1% EDTA, PBS, precipitated by centrifugation, and collected. From the cells collected, RNA was isolated using Isogen (NIPPON GENE) according to the manufacturer's instructions. The amount of HCV RNA contained in the isolated RNA was analyzed by the quantitative RT-PCR method.

### Method of Determining the Minus Strand

The presence of HCV RNA replication was investigated by whether the minus strand in the 5′UTR regin of HCV RNA can be detected in the cells. A specific determination method for the minus strand was carried out similarly to the specific detection method for the minus strand described in the Japanese Unexamined Patent Publication (Kokai) No. 08-187097.

A significant amount of a minus strand RNA could be detected in the cells in which RNA in vitro-synthesized with pLV207TFRepG14 as the template was transfected by the electroporation, and therefore the replication of HCV RNA was confirmed.

### Example 4

### Correlation of the Quantitative Ratio of Truncated RNA to the Total RNA with HCV-Related Diseases

The quantitation of HCV RNA isolated from patient samples was determined as follows. When RNA is determined using 5′UTR as the target, Chiba-S and Chiba-AS primers were used. Using the QauntiTect SYBR Green RT-PCR Kit (QIAGEN), a reaction mixture was prepared under the condition recommended by the manufacturer, transferred to the LightCycler Capillary (Roche Diagnostics), set to the LightCycler (Roche Diagnostics), reacted and PCR products were monitored with time. Appropriately diluted known concentrations of RNA containing in vitro-synthesized HCV 5′UTR were used as the standards. LightCycler software (V3.5.3) was used in analysis.

When RNA was determined with the E2 region as the target, the HC1986S primer and the HC2199-as primer were used. Using the OneStep RT-PCR kit (QIAGEN), a reverse transcription reaction was carried out under the condition recommended by the manufacturer. This reaction mixture was added to a reaction mixture prepared by the LightCycler-FastStart DNA Master SYBR Green I kit (Roche Diagnostics) containing equal amounts of primers, transferred to the LightCycler Capillary (Roche Diagnostics), and set to the LightCycler (Roche Diagnostics). This was subjected to a PCR reaction, and PCR products were monitored with time. Appropriately diluted known concentrations of RNA containing in vitro-synthesized HCV 5′UTR were used as the standards, and using the LightCycler software, the quantitative values were determined.

### Example 5

### Expression of cDNA Encoding a Truncated Sequence in a Mammalian Cell and Analysis of HCV Protein

By inserting an about 8.5 kb fragment obtained by cleaving pLV207TFRepG14 with NotI and XbaI into NotI and XbaI of pcDNA3.1, pcD-LVTRG was obtained. This plasmid DNA was transfected into human kidney-derived cultured cells 293TRex using the Lipofectamine 2000 (Invitrogen). At four hours after DNA transfection, the medium was changed, and the cells were further cultured for 18 hours. Cells were detached from the plate, precipitated by centrifugation, and collected. The cells were disrupted by pipetting in the RIPA buffer, and the supernatant was collected by centrifugation. To the collected supernatant, a ⅓ amount of 3×SDS sample buffer (187.5 mM Tris-HCl [pH 6.8], 6% SDS, 125 mM DTT, 30% glycerol) was added, and heat-treated at 95° C. for 5 minutes.

It was applied on a polyacrylamide gel (Daiichi Kagaku Yakuhin), and was subjected to electrophoresis according to the manufacturer's instructions. After electrophoresis, the proteins were transferred to a PVDF membrane (Millipore) using the Semi-Dry blotter (Sartorius) according to a standard method. After washing the transferred membrane in TTBS (20 mM Tris-HCl [pH 7.5], 150 mM NaCl, 0.1% Tween 20), it was reacted in a 10-fold diluted blocking agent (Milk Diluent/Blocking Solution, Kirkegaad & Perry Laboratories) at room temperature for 2 hours. A primary antibody diluted to a final concentration of 0.3 μg/ml was added thereto, and reacted under shaking at room temperature for 1.5 hour. The reaction mixture was discarded, washed three times in TTBS, and then a 40,000-fold diluted HRP-labelled antibody was added thereto, and reacted under shaking at room temperature for 1 hour.

The reaction mixture was discarded, washed three times in TTBS, and then reacted using the SuperSignal West Pico Chemiluminescent Substrate (Pierce) at room temperature for 5 minutes. The resulting chemiluminescence was detected using the LAS1000 (Fuji Film) or, when the signal was inadequate, exposed to the BioMax Film (Kodak) for detection. As predicted from the sequences of HCV cDNA of pLV207TRG, the core antigen was detected at a position corresponding to the molecular weight of the matured core antigen and the NS3 antigen was detected at a position corresponding to the molecular weight of the matured NS3 antigen using an anti-core monoclonal antibody and an anti-NS3 rabbit antiserum, respectively.

Furthermore, when reacted with an anti-E1 monoclonal antibody, the proteins having a molecular weight close to the normal molecular weight of about 35 kd can be detected, whereas after EndoH-treatment, it changed to a molecular weight of 24 kd. This molecular weight almost agrees with the molecular weight of an amino acid sequence predicted from HCV cDNA of LV207, said molecular weight being calculated from the amino acid sequence of a fusion protein of E1 and NS2. This revealed that E1 and NS2 occur as a fusion protein, and undergo sugar-chain modification. These have shown that the HCV polyprotein encoded by HCV cDNA of LV207 has been cleaved at the same cleavage site as the polyprotein of the FLF type.

### Example 6

### Construction of HCV RNA Replicon and Replication in the Cell

A replicon of the TF type having no neomycin-resistant gene, a drug resistant marker, was constructed. The pSbf-LV207TF plasmid constructed in Example 3 was cleaved with NotI and ClaI to obtain an about 0.7 kb fragment. By inserting this fragment in between the NotI site and the ClaI site of plasmid pLV207TFRepG14 constructed in Example 3, a plasmid pLV207TF was constructed.

With this plasmid cleaved with NotI and XbaI as the template, RNA was synthesized using the Megascript T7 Kit (Ambion). This RNA was purified according to the manufacturer's instructions, and used for transfection into cells.

The purified RNA was transfected by electroporation into the human hepatoma cells Huh7 that had been cultured for 2 days. The cells were detached by treating with trypsin and EDTA, resuspended into a serum-added medium, and trypsin was inactivated. After washing twice in PBS, they were suspended in Cytomix (120 mM potassium chloride, 10 mM potassium phosphate, 5 mM magnesium chloride, 25 mM HEPES, 0.15 mM calcium chloride, 2 mM EGTA, pH 7.6) supplemented 1.25% DMSO, and then about 4×10⁶ cells were transferred to an electroporation cuvette with a gap of 0.4 cm.

After adding 10 μg of RNA into the cuvette, the cells are fully cooled on ice for 5 minutes. Cells were pulsed at 270 V 960 μF using the electroporator (Bio-Rad). They are immediately resuspended into 8 ml of a medium, and seeded in a 12-well plate (22.1 mm in diameter). At 4 hours, 24 hours, 48 hours, 72 hours and 96 hours, the cells were detached with 0.1% EDTA-PBS, and collected by centrifugation. The cell pellet was dissolved in 50 μl of the RIPA buffer (20 mM Tris-HCl [pH 7.5], 150 mM NaCl, 1 mM EDTA, 1% NP40, 0.1% deoxycholate, 0.1% SDS, a complete protease inhibitor cocktail [Roche Diagnostics Corporation]), and After centrifuging at 10 krpm for 5 minutes, the supernatant was collected. The amounts of HCV core antigen in 10 μl of the supernatant was determined using a kit (Fujirebio, Lumipulse) for HCV core antigen.

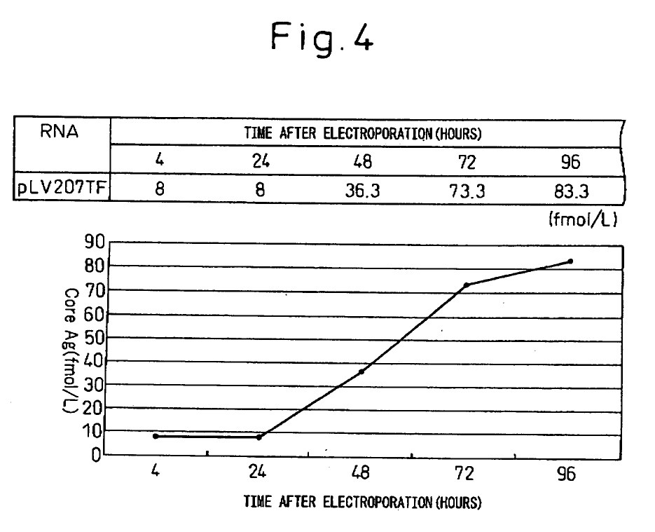

As shown in FIG. 4, the measured value for the core antigen is below the detection limit until 24 hours, it started to increase from 48 hours and was increasing after 96 hours. This indicates that the replicon of TF type of the present invention is replicated in the cells and is replicating the core protein. It demonstrates that the TF genome having the same structure as that replicating in the liver can be replicated in vitro.

### Example 7

### Preparation of the Truncated Form Gene from HCV Samples of the Genotype 2

In Example 2, a truncated form gene was detected from a biopsy sample of a patient with chronic hepatitis by the RT-PCR method, whereas no truncated form gene was detected from samples of the genotype 2 except the sample of BP203. The reason for this was possibly that the sequences of the primers used for detection were designed based on the sequence of the genotype 1.

Thus, based on the sequence of the genotype 2, primers were designed, and with the primers, the detection of the truncated form gene from biopsy samples of patients with chronic hepatitis C of the genotype 2 was attempted.

cDNA synthesis, PCR, cloning of PCR fragments and determination of base sequences were carried out for samples of the genotype 2 shown in Table 1 according to the method of Example 2, except the primers used. Combinations of cDNA synthesis and PCR primers were the following two sets. In primer set A, the primers are 2a_HC3293R for cDNA synthesis, 2a_{—}807S and 2a_{—}3216R for 1st PCR, and 2a_HC835S and 2a_HC3203R for 2nd PCR, and in primer set B, 2a_HC3156R for cDNA synthesis, 2a_{—}807S and 2a_{—}3144R for 1st PCR, and 2a_HC835S and 2a_HC3108R for 2nd PCR. The sequences of the primers are shown below.

| | |
|---|---|
| 2a_HC3293R: | |
| TCTCCATTGGGCTGAACACCACAGGCTCCAC | (SEQ ID NO: 84) |
| 2a_HC3216R: | |
| GGGGAGAGGTGGTCATAGATGTAAGTGCCGG | (SEQ ID NO: 85) |
| 2a_HC3203R: | |
| CATAGATGTAAGTGCCGGTCCACCTGCCTA | (SEQ ID NO: 86) |
| 2a_HC3144R: | |
| CTCCTGCGAGGTGTCTCACCAGGGTACACA | (SEQ ID NO: 87) |
| 2a_HC3108R: | |
| AGCAGAGCGTGAGCTCTGACGAAGTATGG | (SEQ ID NO: 88) |
| 2a_HC835S: | |
| GGAATCTACCCGGTTGCTCTTTTTCTATCTTC | (SEQ ID NO: 89) |
| 2a_HC807S: | |
| CTGGAAGACGGGATAAATTATGCAACAGGGAA | (SEQ ID NO: 90) |

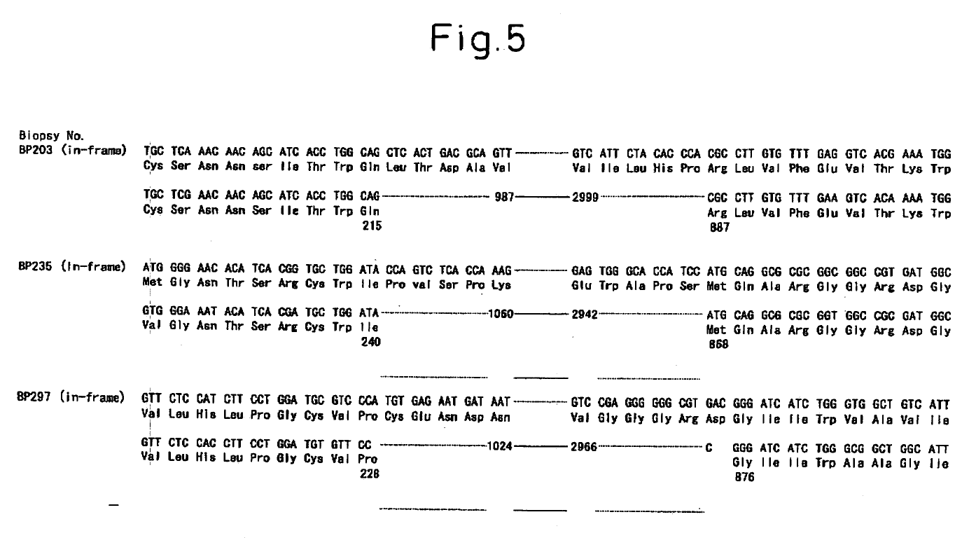

As shown in Table 7, in 6 of 9 samples for primer set A and in 2 of 9 samples for primer set B, genes having deletion were detected. Sequence of these genes indicated that, BP203, BP235 and BP297 had a deletion of about 2 kb in 987-2999 nt, 1060-2945 nt and 1024-2966 nt, respectively, and the typical truncated form genes connected in-frame were observed. The sequence of the deleted region is shown in FIG. 5.

TABLE 7

| | |
|---|---|
| primer set A | primer set B |

| | FL | TF | FL | TF |
|---|---|---|---|---|
| 178 | — | ND | — | ND |
| 201 | — | ND | — | ND |
| 203 | ND | — | ND | ND |
| 235 | ND | — | — | ND |
| 248 | — | — | — | ND |
| 297 | — | — | — | — |
| 298 | — | — | — | — |
| 305 | — | — | — | ND |
| 357 | — | ND | — | ND |

ND: not detected
Primer set A
cDNA: 2a_HC3293R
1st PCR: 2a_HC807Sx2a_HC32l6R
2nd PCR: 2a_HC835Sx2a_HC3203R
Primer set B
cDNA: 2a_HC3156R
1st PCR: 2a_HC807Sx2a_HC3144R
2nd PCR: 2a_HC835Sx2a_HC3108R

### Example 8

### The Detection Rate of the Truncated Form Gene from the Liver Tissue of Patients with Chronic Active Hepatitis and Patients with Hepatic Carcinoma

Summarizing the results of Example 2 and Example 7 revealed that in 6 samples of BP207, BP203, BP325, BP297, BP368 and BP373 among the liver tissues of 24 tissue samples including BP207 from patients with chronic active hepatitis, typical truncated form genes in which an about 2 kb gene from E1 to NS2 is deleted in-frame were detected. On the other hand, from 3 hepatoma tissues, a similar typical truncated form gene was detected in BP1 and BP2. The detection rates of truncated form gene for them were 25% ( 6/24) in patients with chronic hepatitis and 66.6% (⅔) in patients with hepatic carcinoma, and the detection rate increased with the progress from chronic hepatitis to hepatic carcinoma.

Furthermore, the detection of the truncated form gene in 20 plasma samples from asymptomatic carriers was attempted, but no typical truncated form gene was detected. The result indicates that the progress of pathological conditions from chronic hepatitis to cirrhosis to hepatic carcinoma by the infection of hepatitis C virus is related to the presence of the truncated form gene in some way. Thus, the detection of the truncated form gene is considered to be useful as a predictive factor for the progress of pathological conditions.

### Example 9

### Preparation of the Truncated Form Gene from Patients with Fulminant Hepatitis

Preparation of the Truncated Form Gene from Serums of patients with fulminant hepatitis was attempted. From the patient serum, RNA was extracted using ISOGEN-LS (NIPPON GENE CO., LTD.) according to the attached instruction.

In a procedure similar to that used in obtaining genes from BP207 in Example 1, the HCV gene from positions 85 to 9302 was obtained from this RNA. Sequences of 11 clones that were cloned into the pGEM-T Easy vector indicated that they are typical truncated form gene in which positions 922 to 1062, positions 1096 to 1131 and positions 1209 to 2997 were deleted in 10 clones. Though in the rest of one clone, there was no deletion from positions 1096 to 1131, but the clone was typical truncated form gene in which positions 922 to 1062 and positions 1209 to 2997 were deleted.

Furthermore, cDNA in the 5′-untranslated region and the 3′-untranslated region was obtained from the RNA of this patient in the method described in Example 1.

Then, in order to determine the sequence of the end of the 5′-untranslated region, obtainment of the end sequence by the 5′ RACE method was attempted. From patient RNA, using the kit of the 5′ RACE System for Rapid Amplification of cDNA Ends, Version 2.0 (Invitrogen Corporation) according to the attached instruction, the end of the 5′-untranslated region of HCV was obtained. As the antisense primer for cDNA synthesis, Chiba-as was used. cDNA was synthesized by the SuperScript II Reverse Transcriptase, purified by the S.N.A.P. column, and then purified cDNA was subjected to TdT-tailing reaction to add dCTP. The cDNA was subjected to the 1st PCR using the 5′ RACE Abridged Anchor primer, KY78 primer attached to the kit: 5′-CTCGCAAGCACCCTATCAGCCAGT-3′ (SEQ ID NO: 91) and LA Taq (TAKARA). With a portion of the PCR product as the template, the 2nd PCR was carried out using the UAP primer, the KM2 primer attached to the kit: 5′-AGGCATTGAGCGGGTTTATC-3′ (SEQ ID NO: 92) and LA Taq (TAKARA) to obtain a PCR product. This PCR product was cloned into the pGEM-T Easy vector, and the sequence was determined. As a result, this truncated form gene had from the sequence at position 1 to the 5′-untranslated region similarly to the standard full-length HCV gene.

Furthermore, in order to determine the sequence of the end of the 3′-untranslated region, obtaining the end sequence by the 3′ RACE method was attempted. First, using the Poly(A) Tailing Kit (Ambion, Inc.) according to the attached instruction, Poly(A) was added to the RNA from the patient. From the Poly(A)-added RNA, using dT-Adp primer: 5′-CTAGACTCGAGTCGACATCGTTTTTTTTTTTTTTTTTT-3′ (SEQ ID NO: 93), cDNA was synthesized in a manner similar to the procedure for cDNA synthesis described in Example 1. With this cDNA as the template, the 1st PCR was carried out using 3UTR-1F primer: 5′-ATCTTAGCCCTAGTCACGGC-3′ (SEQ ID NO: 94) and the Adp primer: 5′-CTAGACTCGAGTCGACATCG-3′ (SEQ ID NO: 95), and the 2nd PCR was carried out using XR58F: 5′-CTAGCTGTGAAAGGTCCGTGAGCCGCATGA-3′ (SEQ ID NO: 96) and the Adp primer (SEQ ID NO: 95) using LA Taq (TAKARA). This PCR product was cloned into the pGEM-T Easy vector, and the sequence was determined. As a result, the 3′-end of this truncated form gene had a 3′-end similar to that in the standard full-length HCV gene.

### INDUSTRIAL APPLICABILITY

As an application example of the present invention, the replicon replication system can be used in drug screening for the development of therapeutic agents for hepatitis C virus. This system can also be used in the efficacy evaluation and the production of therapeutic agents. Also, the detection system of the TF genome can be used as a pathological marker for hepatitis C and is useful as a diagnostic reagent.

1. A truncated form hepatitis C virus gene wherein part of the E1 protein-coding region, the E2 protein-coding region, the P7 protein-coding region and part of the NS2 protein-coding region have been deleted while retaining the translation frame in the hepatitis C virus gene.

2. The truncated form hepatitis C virus gene according to claim 1, said gene having all or part of a region encoding from the 5′-untranslated region to the core protein which is a structural protein and all or part of a region encoding from a region encoding two transmembrane domains at the latter part of NS2 which is a nonstructural protein to the 3′-untranslated region.

3. The truncated form hepatitis C virus gene according to claim 1, said gene having all or part of No. 1 to No. 914 and all or part of No. 3001 and after of the nucleic acid sequence of the hepatitis C virus gene.

4. The replicon gene according to claim 1 that autonomously replicates itself in the cell.

5. The replicon gene according to claim 4 to which a selection marker gene has been connected.

6. The cell in which the above replicon according to claim 4 is replicated.

7. A method of screening or evaluating the efficacy of drugs using the cell according to claim 6.

8. A cell retaining a vector having integrated the gene according to claim 1 and that is expressing the protein.

9. A method of diagnosing HCV using the cell according to claim 6 in which the replicon is being replicated or the protein produced by the cell.

10. A method of detecting the truncated form gene of hepatitis C virus using a method that detects the deletion of a gene.

11. A method of detecting or quantitating the truncated form gene by amplifying the truncated form gene by PCR with primers designed from sequences of nucleic acids 1-914 and 3001 and after of the hepatitis C virus gene.

12. A method of determining the quantitative ratio by quantitating the gene at the common region of the truncated form gene and the full-length form gene, and quantitating the gene at the deleted region of the truncated form gene.

13. Hepatitis C virus particles or hepatitis C virus-like particles retaining the gene described in claim 1.

14. A polyprotein of the hepatitis C virus produced by the gene according to claim 1 and a protein processed from the polyprotein.

15. An antibody that specifically recognizes the protein according to claim 14.
